SEQUENCE LISTING (10 sequences)

SEQ ID NO: 1 - DNA, 1212 bp, Homo sapiens
  source 1..1212: mol_type = other DNA
atggcatcagaactggcaatgagcaactccgacctgcccaccagtcccctggccatggaatatgttaatgacttcgatctgatgaagtttgaagtgaaaaaggaaccggtggagaccgaccgcatcatcagccagtgcggccgtctcatcgccgggggctcgctgtcctccacccccatgagcacgccgtgcagctcggtgcccccttcccccagcttctcggcgcccagcccgggctcgggcagcgagcagaaggcgcacctggaagactactactggatgaccggctacccgcagcagctgaaccccgaggcgctgggcttcagccccgaggacgcggtcgaggcgctcatcagcaacagccaccagctccagggcggcttcgatggctacgcgcgcggggcgcagcagctggccgcggcggccggggccggtgccggcgcctccttgggcggcagcggcgaggagatgggccccgccgccgccgtggtgtccgccgtgatcgccgcggccgccgcgcagagcggcgcgggcccgcactaccaccaccaccaccaccacgccgccggccaccaccaccacccgacggccggcgcgcccggcgccgcgggcagcgcggccgcctcggccggtggcgctgggggcgcgggcggcggtggcccggccagcgctgggggcggcggcggcggcggcggcggcggaggcggcgggggcgcggcgggggcggggggcgccctgcacccgcaccacgccgccggcggcctgcacttcgacgaccgcttctccgacgagcagctggtgaccatgtctgtgcgcgagctgaaccggcagctgcgcggggtcagcaaggaggaggtgatccggctgaagcagaagaggcggaccctgaaaaaccgcggctatgcccagtcctgccgcttcaagagggtgcagcagagacacgtcctggagtcggagaagaaccagctgctgcagcaagtcgaccacctcaagcaggagatctccaggctggtgcgcgagagggacgcgtacaaggagaaatacgagaagttggtgagcagcggcttccgagaaaacggctcgagcagcgacaacccgtcctctcccgagtttttcataactgagcccactcgcaagttggagccatcagtgggatacgccacattttggaagccccagcatcgtgtacttaccagtgtgttcacaaaatga

SEQ ID NO: 2 - protein, 403 aa, Homo sapiens
MASELAMSNSDLPTSPLAMEYVNDFDLMKFEVKKEPVETDRIISQCGRLIAGGSLSSTPMSTPCSSVPPSPSFSAPSPGSGSEQKAHLEDYYWMTGYPQQLNPEALGFSPEDAVEALISNSHQLQGGFDGYARGAQQLAAAAGAGAGASLGGSGEEMGPAAAVVSAVIAAAAAQSGAGPHYHHHHHHAAGHHHHPTAGAPGAAGSAAASAGGAGGAGGGGPASAGGGGGGGGGGGGGGAAGAGGALHPHHAAGGLHFDDRFSDEQLVTMSVRELNRQLRGVSKEEVIRLKQKRRTLKNRGYAQSCRFKRVQQRHVLESEKNQLLQQVDHLKQEISRLVRERDAYKEKYEKLVSSGFRENGSSSDNPSSPEFFITEPTRKLEPSVGYATFWKPQHRVLTSVFTK

SEQ ID NO: 3 - DNA, 1122 bp, Homo sapiens
  source 1..1122: mol_type = other DNA
atggcatcagaactggcaatgagcaactccgacctgcccaccagtcccctggccatggaatatgttaatgacttcgatctgatgaagtttgaagtgaaaaaggaaccggtggagaccgaccgcatcatcagccagtgcggccgtctcatcgccgggggctcgctgtcctccacccccatgagcacgccgtgcagctcggtgcccccttcccccagcttctcggcgcccagcccgggctcgggcagcgagcagaaggcgcacctggaagactactactggatgaccggctacccgcagcagctgaaccccgaggcgctgggcttcagccccgaggacgcggtcgaggcgctcatcagcaacagccaccagctccagggcggcttcgatggctacgcgcgcggggcgcagcagctggccgcggcggccggggccggtgccggcgcctccttgggcggcagcggcgaggagatgggccccgccgccgccgtggtgtccgccgtgatcgccgcggccgccgcgcagagcggcgcgggcccgcactaccaccaccaccaccaccacgccgccggccaccaccaccacccgacggccggcgcgcccggcgccgcgggcagcgcggccgcctcggccggtggcgctgggggcgcgggcggcggtggcccggccagcgctgggggcggcggcggcggcggcggcggcggaggcggcgggggcgcggcgggggcggggggcgccctgcacccgcaccacgccgccggcggcctgcacttcgacgaccgcttctccgacgagcagctggtgaccatgtctgtgcgcgagctgaaccggcagctgcgcggggtcagcaaggaggaggtgatccggctgaagcagaagaggcggaccctgaaaaaccgcggctatgcccagtcctgccgcttcaagagggtgcagcagagacacgtcctggagtcggagaagaaccagctgctgcagcaagtcgaccacctcaagcaggagatctccaggctggtgcgcgagagggacgcgtacaaggagaaatacgagaagttggtgagcagcggcttccgagaaaacggctcgagcagcgacaacccgtcctctcccgagtttttcatgtga

SEQ ID NO: 4 - protein, 373 aa, Homo sapiens
MASELAMSNSDLPTSPLAMEYVNDFDLMKFEVKKEPVETDRIISQCGRLIAGGSLSSTPMSTPCSSVPPSPSFSAPSPGSGSEQKAHLEDYYWMTGYPQQLNPEALGFSPEDAVEALISNSHQLQGGFDGYARGAQQLAAAAGAGAGASLGGSGEEMGPAAAVVSAVIAAAAAQSGAGPHYHHHHHHAAGHHHHPTAGAPGAAGSAAASAGGAGGAGGGGPASAGGGGGGGGGGGGGGAAGAGGALHPHHAAGGLHFDDRFSDEQLVTMSVRELNRQLRGVSKEEVIRLKQKRRTLKNRGYAQSCRFKRVQQRHVLESEKNQLLQQVDHLKQEISRLVRERDAYKEKYEKLVSSGFRENGSSSDNPSSPEFFM

SEQ ID NO: 5 - DNA, 1152 bp, Homo sapiens
  source 1..1152: mol_type = other DNA
atggcatcagaactggcaatgagcaactccgacctgcccaccagtcccctggccatggaatatgttaatgacttcgatctgatgaagtttgaagtgaaaaaggaaccggtggagaccgaccgcatcatcagccagtgcggccgtctcatcgccgggggctcgctgtcctccacccccatgagcacgccgtgcagctcggtgcccccttcccccagcttctcggcgcccagcccgggctcgggcagcgagcagaaggcgcacctggaagactactactggatgaccggctacccgcagcagctgaaccccgaggcgctgggcttcagccccgaggacgcggtcgaggcgctcatcagcaacagccaccagctccagggcggcttcgatggctacgcgcgcggggcgcagcagctggccgcggcggccggggccggtgccggcgcctccttgggcggcagcggcgaggagatgggccccgccgccgccgtggtgtccgccgtgatcgccgcggccgccgcgcagagcggcgcgggcccgcactaccaccaccaccaccaccacgccgccggccaccaccaccacccgacggccggcgcgcccggcgccgcgggcagcgcggccgcctcggccggtggcgctgggggcgcgggcggcggtggcccggccagcgctgggggcggcggcggcggcggcggcggcggaggcggcgggggcgcggcgggggcggggggcgccctgcacccgcaccacgccgccggcggcctgcacttcgacgaccgcttctccgacgagcagctggtgaccatgtctgtgcgcgagctgaaccggcagctgcgcggggtcagcaaggaggaggtgatccggctgaagcagaagaggcggaccctgaaaaaccgcggctatgcccagtcctgccgcttcaagagggtgcagcagagacacgtcctggagtcggagaagaaccagctgctgcagcaagtcgaccacctcaagcaggagatctccaggctggtgcgcgagagggacgcgtacaaggagaaatacgagaagttggtgagcagcggcttccgagaaaacggctcgagcagcgacaacccgtcctctcccgagtttttcatgtacccaagggaatcctctacatcagtgatgtga

SEQ ID NO: 6 - protein, 383 aa, Homo sapiens
MASELAMSNSDLPTSPLAMEYVNDFDLMKFEVKKEPVETDRIISQCGRLIAGGSLSSTPMSTPCSSVPPSPSFSAPSPGSGSEQKAHLEDYYWMTGYPQQLNPEALGFSPEDAVEALISNSHQLQGGFDGYARGAQQLAAAAGAGAGASLGGSGEEMGPAAAVVSAVIAAAAAQSGAGPHYHHHHHHAAGHHHHPTAGAPGAAGSAAASAGGAGGAGGGGPASAGGGGGGGGGGGGGGAAGAGGALHPHHAAGGLHFDDRFSDEQLVTMSVRELNRQLRGVSKEEVIRLKQKRRTLKNRGYAQSCRFKRVQQRHVLESEKNQLLQQVDHLKQEISRLVRERDAYKEKYEKLVSSGFRENGSSSDNPSSPEFFMYPRESSTSVM

SEQ ID NO: 7 - DNA, 1113 bp, Mus musculus
  source 1..1113: mol_type = other DNA
atggcttcagaactggcaatgaacaattccgacctgcccaccagtcccctggccatggaatatgttaatgacttcgatctgatgaagtttgaagtgaaaaaggaaccggtggagaccgaccgcatcatcagccagtgcggccgtctcatcgccgggggctcgctgtcctccacccccatgagcacgccctgcagctcggtgcccccgtcccccagcttctcggcgcccagcccgggctcgggcagcgaacagaaggcgcacctggaagactactactggatgaccggctacccgcagcagctcaacccggaggcgctgggcttcagcccggaggacgcggtcgaggcgctcatcagcaacagccaccagctccagggtggcttcgatggctatgcgcggggagcgcagcagctggccgcggcagcgggggccggcgccggcgcctccctgggcggcagcggcgaggagatgggccccgccgccgccgtggtgtccgccgtgatcgccgcggccgccgcgcagagcggcgcggcaccccactaccatcaccaccaccaccacgccgcggggcaccaccaccatccgacggccggcgccccgggagccgcgggcggcgcgtctgcctctgcgagcggcgcgggtggcgcgggcggcggtggcccggccagcgccgggggcggcggcggcggaggcggcggcgggggcacggcgggggcggggggcgccctgcacccgcaccatgccgcgggcggcctgcacttcgacgaccgcttctcggacgagcagttggtgaccatgtcggtgcgcgagctgaaccggcagctgcgcggggtcagcaaggaggaggtgatccgactgaagcagaagaggcggaccctgaaaaaccgcggctatgcccagtcctgccgcttcaagagggtgcagcagagacacgtcctggagtcggagaagaaccagctgctgcagcaggtagaccacctcaagcaggagatctccaggctggtgcgcgaaagggacgcctacaaggagaaatacgagaagctggtgagcaacggcttccgagaaaacggctcgagcagcgacaacccttcctctcccgaatttttcatgtga

SEQ ID NO: 8 - protein, 370 aa, Mus musculus
MASELAMNNSDLPTSPLAMEYVNDFDLMKFEVKKEPVETDRIISQCGRLIAGGSLSSTPMSTPCSSVPPSPSFSAPSPGSGSEQKAHLEDYYWMTGYPQQLNPEALGFSPEDAVEALISNSHQLQGGFDGYARGAQQLAAAAGAGAGASLGGSGEEMGPAAAVVSAVIAAAAAQSGAAPHYHHHHHHAAGHHHHPTAGAPGAAGGASASASGAGGAGGGGPASAGGGGGGGGGGGTAGAGGALHPHHAAGGLHFDDRFSDEQLVTMSVRELNRQLRGVSKEEVIRLKQKRRTLKNRGYAQSCRFKRVQQRHVLESEKNQLLQQVDHLKQEISRLVRERDAYKEKYEKLVSNGFRENGSSSDNPSSPEFFM

SEQ ID NO: 9 - DNA, 1143 bp, Mus musculus
  source 1..1143: mol_type = other DNA
atggcttcagaactggcaatgaacaattccgacctgcccaccagtcccctggccatggaatatgttaatgacttcgatctgatgaagtttgaagtgaaaaaggaaccggtggagaccgaccgcatcatcagccagtgcggccgtctcatcgccgggggctcgctgtcctccacccccatgagcacgccctgcagctcggtgcccccgtcccccagcttctcggcgcccagcccgggctcgggcagcgaacagaaggcgcacctggaagactactactggatgaccggctacccgcagcagctcaacccggaggcgctgggcttcagcccggaggacgcggtcgaggcgctcatcagcaacagccaccagctccagggtggcttcgatggctatgcgcggggagcgcagcagctggccgcggcagcgggggccggcgccggcgcctccctgggcggcagcggcgaggagatgggccccgccgccgccgtggtgtccgccgtgatcgccgcggccgccgcgcagagcggcgcggcaccccactaccatcaccaccaccaccacgccgcggggcaccaccaccatccgacggccggcgccccgggagccgcgggcggcgcgtctgcctctgcgagcggcgcgggtggcgcgggcggcggtggcccggccagcgccgggggcggcggcggcggaggcggcggcgggggcacggcgggggcggggggcgccctgcacccgcaccatgccgcgggcggcctgcacttcgacgaccgcttctcggacgagcagttggtgaccatgtcggtgcgcgagctgaaccggcagctgcgcggggtcagcaaggaggaggtgatccgactgaagcagaagaggcggaccctgaaaaaccgcggctatgcccagtcctgccgcttcaagagggtgcagcagagacacgtcctggagtcggagaagaaccagctgctgcagcaggtagaccacctcaagcaggagatctccaggctggtgcgcgaaagggacgcctacaaggagaaatacgagaagctggtgagcaacggcttccgagaaaacggctcgagcagcgacaacccttcctctcccgaatttttcatgtacccaagggactcctctacatctgtgatgtga

SEQ ID NO: 10 - protein, 300 aa, Mus musculus
GSEQKAHLEDYYWMTGYPQQLNPEALGFSPEDAVEALISNSHQLQGGFDGYARGAQQLAAAAGAGAGASLGGSGEEMGPAAAVVSAVIAAAAAQSGAAPHYHHHHHHAAGHHHHPTAGAPGAAGGASASASGAGGAGGGGPASAGGGGGGGGGGGTAGAGGALHPHHAAGGLHFDDRFSDEQLVTMSVRELNRQLRGVSKEEVIRLKQKRRTLKNRGYAQSCRFKRVQQRHVLESEKNQLLQQVDHLKQEISRLVRERDAYKEKYEKLVSNGFRENGSSSDNPSSPEFFMYPRDSSTSVM

(19) United States
(12) Patent Application Publication
(10) Pub. No.: US 2025/0222037 A1
(43) Pub. Date: Jul. 10, 2025

(54) INDUCED LIVER SINUSOIDAL ENDOTHELIAL CELLS (iLSECs) AND METHODS FOR SUPPORTING HEPATOCYTES USING THE iLSECs

(71) Applicant: Cornell University, Ithaca, NY (US)

(72) Inventors: Shahin RAFII, Ithaca, NY (US); Jesus Maria GOMEZ-SALINERO, New York, NY (US)

(73) Assignee: Cornell University, Ithaca, NY (US)

(21) Appl. No.: 18/852,772

(22) PCT Filed: Mar. 30, 2023
(86) PCT No.: PCT/US2023/065138

§ 371 Date: Sep. 30, 2024

Related U.S. Application Data
(60) Provisional application No. 63/325,758, filed on Mar. 31, 2022.

(51) Int. Cl. A61K 35/407; C12N 5/071

(52) U.S. Cl. CPC A61K 35/407; C12N 5/067; C12N 2502/99

(57) ABSTRACT

The present disclosure is directed to an isolated population of induced liver sinusoidal endothelial cells (ILSECs) and methods of generating the iLSECs. The peri-natal induction of the transcription factor c-Maf is a critical switch for the sinusoidal identity determination. Enforced and enhanced expression of c-Maf in generic human endothelial cells switches on a liver sinusoidal transcriptional program that maintains hepatocyte function. The present disclosure provides methods of coculturing hepatocytes with the iLSECs. The present disclosure also provides methods of treating liver diseases using the co-cultured hepatocytes and iLSECs.

### CROSS REFERENCE TO RELATED APPLICATION

This application claims the benefit of priority from U.S. Provisional Application No. 63/325,758, filed on Mar. 31, 2022, the entire content of which is incorporated herein by reference.

### INCORPORATION BY REFERENCE OF SEQUENCE LISTING

The Sequence Listing in an XML format, named as 39351WO_9288_04_PC_SequenceListing of 17 KB, created on Mar. 29, 2023, and submitted to Patent Center, is incorporated herein by reference.

### BACKGROUND

The vascular system encompasses a heterogeneous pool of specialized endothelial cells (ECs) that anastomose arteries to veins and establishes functional and structural organotypic diversity (Harvey et al. (2004) Curr. Opin. Genet. Dev. 14:499-505). This angiodiversity is achieved by the unique capacity of ECs to customize and meet the cellular and metabolic demands of each org. In addition to inter-organ specific heterogeneity, endothelial progenitor cells acquire remarkable intra-organ diversity with certain organs, such as the liver, ultimately arborizing with differentiated specialized ECs (Augustin et al. (2017) Science 357: caal2379; Gomez-Salinero et al. (2018) Science 362, 1116-1117; Jakab et al. (2020) Development 147: dev146621; Rafii et al. (2016) Nature 529:316-325). Tissue-specific microvascular ECs also instruct neighboring cells and adapt to metabolic demands during homeostasis, regeneration and aging (Augustin et al. (2017) Science 357: caal2379; Rafii et al. (2016) Nature 529:316-325). The capacity of ECs to meet these tissue-specific diversified functions is achieved by actively co-adapting to their microenvironment by deploying distinct angiocrine factors to maintain homeostasis and regeneration, or mal-adapt to support tumorigenesis (Cao et al. (2017) Sci. Transl. Med. 9: eaai8710; Dejana et al. (2017) Nat. Commun. 8:14361; Ding et al. (2010) Nature 468:310-315; Ding et al. (2011) Cell 147:539-553; Tavora et al. (2020) Nature 586:299-304). While the transcriptional networks regulating arterial-venous specification have been extensively described, the molecular determinants of intra-organ microvascular EC diversity, such as zonation within liver capillaries (termed sinusoids) remain to be fully characterized.

Liver vasculatures adapt to various developmental, homeostatic and pathophysiological processes, including liver regeneration, fibrosis and cancer (Cao et al. (2017) Sci. Transl. Med. 9: eaai8710; Ding et al. (2010) Nature 468:310-315; de Haan et al. (2020) Am. J. Physiol. Gastrointest. Liver Physiol. 318: G803-G815; Halpern et al. (2018) Nat. Biotechnol. 36:962-970; Lotto et al. (2020) Cell 183:702-716.e14; MacParland et al. (2018) Nat. Commun. 9:4383; Rafii et al. (2016) Nature 529:316-325; Sharma et al. (2020) Cell 183:377-394.e21). Furthermore, liver endothelium is organized in a patterned hepatocyte co-zonation gradient that regulates liver functionalization in part by angiocrine release of Rspo3 and Wnt9b from the Central Vein (Halpern et al. (2018) Nat. Biotechnol. 36:962-970; Planas-Paz et al. (2016) Nat. Cell Biol. 18:467-479; Rocha et al. (2015) Cell Rep. 13:1757-1764). Similarly, the liver sinusoids secrete Wnt2, Hgf, and other angiocrine factors that modulate liver homeostasis and regeneration (Ding et al. (2010) Nature 468:310-315, Ding et al. (2014) Nature 505:97-102; Rafii et al. (2016) Nature 529:316-325). While Gata4 and Bmp9 have been identified as regulators of the sinusoidal signature (Desroches-Castan et al. (2019a) Hepatology 70:1392-1408; Géraud et al. (2017) J. Clin. Invest. 127:1099-1114; Winkler et al. (2021) J. Hepatol. 74:380-393), the mechanism by which differentiated liver vasculature acquires its diverse attributes is still undefined.

### SUMMARY

The present disclosure resolves intra-organ vascular heterogeneity by performing single-cell RNA sequencing (scRNA-seq) of liver endothelium from early fetal to late postnatal development. The present disclosure shows that acquisition of the sinusoidal identity is initiated during early development and is fully established postnatally. The present disclosure identifies induction of the transcription factor c-Maf as a critical driver of liver sinusoidal maturation. Absence of c-Maf impairs hepatic sinusoidal specification, aberrantly expands postnatal liver hematopoiesis, and increases the fibrotic damage induced by chemical insult in adult mouse liver. Notably, enhanced expression of c-Maf induces sinusoidal identity in human generic endothelium. Thus, c-Maf represent an inducible specification factor for liver sinusoids and set the stage to devise future approaches for therapeutic liver repair.

There is another transcription factor, Gata4, that has been previously described to have a similar phenotype in endothelial cells. However, its enhanced expression does not upregulate as many genes as MAF does, and the phenotype of the cells is not as consistent as with the enhanced expression of MAF. Moreover, it is known that the use of Gata4 increases the expression of inflammatory genes and requires other genes to compensate for that.

One aspect of the present disclosure is directed to an isolated population of induced liver sinusoidal endothelial cells (ILSECs), wherein the isolated population comprises iLSECs that express a c-Maf protein from an exogenous nucleic acid and express at the cell surface at least two markers selected from the group consisting of CD26, CD36, and CD14.

In some embodiments, the iLSECs express CD26, CD36, and CD14. In some embodiments, the iLSECs also express MRC1 at the cell surface. In some embodiments, the ILSECs also express VE-Cadherin.

In some embodiments, the iLSECs are made by expressing a c-Maf protein from an exogenous nucleic acid in a population of endothelial cells in culture.

In some embodiments, the exogenous nucleic acid is a viral vector comprising a c-Maf coding nucleic acid. In some embodiments, the viral vector is a lentiviral vector or an AAV vector. In some embodiments, the exogenous nucleic acid is a modified RNA (modRNA) encoding c-Maf.

In some embodiments, the population of endothelial cells are endothelial cells without a liver signature. In some embodiments, the endothelial cells are selected from the group consisting of human umbilical vein endothelial cells (HUVECs), adipose-derived endothelial cells, organ-specific endothelial cells, endothelial stem cells, and endothelial progenitor cells, and wherein the endothelial cells are autologous or allogeneic. In some embodiments, the organ-specific endothelial cells are selected from the group consisting of heart-specific endothelial cells, muscle-specific endothelial cells, kidney-specific endothelial cells, testis-specific endothelial cells, ovary-specific endothelial cells, lymphoid-specific endothelial cells, pancreas-specific endothelial cells, brain-specific endothelial cells, lung-specific endothelial cells, bone marrow-specific endothelial cells, spleen-specific endothelial cells, large intestine-specific endothelial cells, small intestine-specific endothelial cells, and ovary or testicular endothelial cells. In some embodiments, the endothelial cells are endothelial cells which have been isolated from the liver as liver-specific endothelial cells and cultured in vitro after the isolation. Liver-specific endothelial cells may lose their liver signature as a result of an in vitro culture and can be converted to iLSECs by enhanced expression of c-Maf.

In some embodiments, the c-Maf protein is human c-Maf or mouse c-Maf.

In some embodiments, the isolated population of iLSECs supports a co-culture with hepatocytes for at least 20 days, at least 25 days, at least 26 days, at least 27 days, at least 28 days, at least 29 days, at least 30 days, or longer (e.g., up to 40 or 50 days).

Another aspect of the disclosure is directed to a method of producing an isolated population of induced liver sinusoidal endothelial cells (ILSECs). The method comprises, providing a population of endothelial cells, expressing a c-Maf protein from an exogenous nucleic acid in the population of endothelial cells in culture, selecting a population of cells that express at the cell surface at least two markers selected from the group consisting of CD26, CD36, and CD14, thereby obtaining the isolated population of ILSECs.

In some embodiments, the cells selected express at the cell surface all three markers of CD26, CD36, and CD14. In some embodiments, the cells selected also express MRC1 at the cell surface. In some embodiments, the cells selected also express VE-Cadherin.

In some embodiments, the exogenous nucleic acid is a viral vector comprising a c-Maf coding sequence. In some embodiments, the viral vector is a lentiviral vector or an AAV vector. In some embodiments, the exogenous nucleic acid is a modified RNA (modRNA) coding for c-Maf.

In some embodiments, the population of endothelial cells are endothelial cells without a liver signature. In some embodiments, the endothelial cells used in the method are selected from the group consisting of human umbilical vein endothelial cells (HUVECs), adipose-derived endothelial cells, organ-specific endothelial cells, endothelial stem cells, and endothelial progenitor cells. The endothelial cells are autologous or allogeneic. In some embodiments, the organ-specific endothelial cells used in the above-mentioned method, are selected from the group consisting of heart-specific endothelial cells, muscle-specific endothelial cells, kidney-specific endothelial cells, testis-specific endothelial cells, ovary-specific endothelial cells, lymphoid-specific endothelial cells, pancreas-specific endothelial cells, brain-specific endothelial cells, lung-specific endothelial cells, bone marrow-specific endothelial cells, spleen-specific endothelial cells, large intestine-specific endothelial cells, small intestine-specific endothelial cells, and ovary or testicular endothelial cells. In some embodiments, the endothelial cells are endothelial cells which have been isolated from the liver as liver-specific endothelial cells and cultured in vitro after the isolation. Liver-specific endothelial cells may lose their liver signature as a result of an in vitro culture and can be converted to iLSECs by enhanced expression of c-Maf.

In some embodiments, the c-Maf protein is human c-Maf or mouse c-Maf.

In some embodiments, a population of endothelial cells is induced for enhanced expression of c-Maf for a period of 5-9 days, e.g., 6 days, 7 days, or 8 days. In some embodiments, enhanced expression of c-Maf can be achieved using a doxycycline induced expression system; and in such embodiments, doxycycline can be added to the culture media and the endothelial cells are cultured for a period of 5-9 days, e.g., 6 days, 7 days, or 8 days. Therefore, the cells that co-express at least two markers selected from the group consisting of CD26, CD36, and CD14, and optionally also MRC1, are isolated as iLSEC.

Another aspect of the disclosure is directed to a method of maintaining hepatocytes. The method comprises culturing hepatocytes in the presence of an isolated population of iLSECs disclosed herein.

In some embodiments, the culturing is done for at least 20 days to at least 30 days, e.g., at least 28 days, and up to, e.g., 40 to 50 days.

iLSECs and hepatocytes can be combined in any suitable tissue culture device to allow for interaction between the cells and formation of spheroids. Preferably iLSECs and hepatocytes are combined and co-cultured in the presence of lethally irradiated fibroblast. The spheroids formed can be maintained in medium that contains fetal bovine serum (FBS) (optional), Insulin-Transferrin-Selenium (ITS) at 1× concentration, and HGF at 50 ng/ml.

In some embodiments, the co-culturing is carried out at an oxygen tension between 1% and 20%, e.g., at 20%.

In some embodiments, the culturing occurs in a bioreactor, a tissue culture plate or a microfluidic device.

In some embodiments, the culturing is performed in a decellularized matrix, a 3D scaffold, or a bioengineer matrix for the transplantation.

Another aspect of the disclosure is directed to a method of treating a damaged liver or a liver disease in a subject comprising administering to the subject an isolated population of iLSECs.

In some embodiments, the administration to the subject comprises administration of hepatocytes together with the isolated population of ILSECs.

In some embodiments, the administering is achieved by a method selected from the group consisting of surgical or catheter implantation, subcutaneous injection, and infusion through an intravascular route. In some embodiments, the cells are administered to a site in the omentum or in the liver under the liver capsule through, e.g., implantation or subcutaneous injection.

In some embodiments, the liver disease is selected from the group consisting of acetaminophen toxicity, acute liver failure, alcoholic liver disease, liver cancer, cirrhosis, liver cyst, non-alcoholic fatty liver disease (NAFLD), and liver fibrosis.

Another aspect of the disclosure is directed to a method of treating a subject suffering from a liver disease. The method comprises administering to the subject hepatocytes which have been cultured in the presence of an isolated population of iLSECs as described herein. In some embodiments, the iLSECs from the coculture are administered with the hepatocytes.

In some embodiments, the administering is achieved by surgical implantation, catheter implantation, subcutaneous injection, or infusion through an intravascular route. In some embodiments, the cells are administered to a site in the omentum or in the liver under the liver capsule through, e.g., implantation or subcutaneous injection.

In some embodiments, the liver disease is selected from the group consisting of acetaminophen toxicity, acute liver failure, alcoholic liver disease, liver cancer, cirrhosis, liver cyst, non-alcoholic fatty liver disease (NAFLD), and liver fibrosis.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fec.

FIGS. 1A-G. Liver vasculature progenitor diversification is acquired during transition from fetal to postnatal development. (A) UMAP from endothelial cells (ECs) identified by single cell RNA-seq analysis (scRNA-seq) of sorted liver ECs (CD45^{neg}CD31⁺) at fetal and postnatal timepoints E12, E14, E16, E18, P2, P8, P15, and P30. Colors are assigned based on the sample timepoint as indicated in the upper panel. (B) UMAP labeling the different EC populations identified from the scRNA-seq analysis from (A). Cavin3⁺: Cv3, Portal Vein: PV, Central Vein: CV, Fetal sinusoidal EC populations: FS1-5, Postnatal sinusoidal EC populations PS1-5, Proliferating ECs: P1-3, Cxc110 high expressing ECs: C^{high}. (C) Identification of specific markers associated to the EC populations identified from the single cell analysis. (D) Transcriptional similarity analysis within Portal Vein, Central Vein and Sinusoids cell groups during Fetal and Postnatal development, as calculated by Pearson correlation coefficient (PCC) in the principal component space. (E) Vascular populations identified from the single cell analysis ordered using pseudotime and plotted based on their predicted order. (F) Proportion of the contribution of Cavin3⁺ (Cv3), Portal Vein (PV), Central Vein (CV), or all sinusoids(S) per timepoint. Colors indicate the timepoint. (G) Diagram of the development of the liver vascular system following the observations from single cell analysis. During early development (E12) the liver bud is infiltrated by the Vitellin Vein, Umbilical Vein (HUVEC) and Sinus Venosus. After this infiltration the endothelium differentiates into the Portal Vein and the sinusoids. The Central Vein population gets differentiated later in development, starting primarily at E18. During the progressive development of the liver, the sinusoidal transcriptome transitions from a fetal to an adult state. Colors are based on the identified populations in (B).

FIGS. 2A-J. Liver vascular development is associated with a bi-sequential specification within a fetal to postnatal transition. (A) Pseudotime analysis of the endothelial cell populations. Arrows show the directionality of changes, associated with the transition from fetal to postnatal development. (B) Identification of the top 300 genes contributing to the changes over pseudotime. Colors correspond to an increase in changes. (C) Gene ontology analysis of the genes contributing to fetal or postnatal pseudotime transition. (D) Analysis of the expression velocity of Mrc1 and Fegr2b measured as the ratio of spliced versus unspliced RNA over time, and the expression levels of these genes, derived from single cell RNA-seq. (E-F) Analysis of the percentage of ECs positive for Mrc1 (E) and Fcgr2b (F), of the ECs across the fetal E12, E14, E16, E18 and Adult. Data represents n≥8. t-test analysis was performed comparing E12 to each individual timepoint, *p<0.05, ***p<0.001, ****p<0.0001. (G) Average expression of the transcription factors driving the postnatal vascular transition in FIG. 2B within the endothelial cell (EC) of each organ obtained from the Tabula Muris database. Colors represent the average expression, while size of the dot represents the percentage of cells where the expression was detected. (H) Violin plot showing expression of c-Maf within the EC clusters per timepoint from the single cell analysis. (I-J) Flow cytometry analysis of the expression levels of c-Maf-Mrc1 (G, H), and c-Maf-Fcg2b (I, J) in the liver ECs, n≥5. Colors indicate the quadrant from the flow cytometry gating.

FIGS. 3A-G. c-Maf choreographs the acquisition of sinusoidal attributes during maturation of immature liver capillaries. (A) c-Maf^{flox/flox} mice were crossed with VEcadherin^{Cre-Ert2} mice, induced with tamoxifen from E12 to E14, and analyzed at the E16 developmental timepoint. Fetal mice and livers from control and c-Maf^{ΔEC} are shown. (B) Flow cytometry analysis of the vasculature showing the deletion specificity of c-Maf within the ECs (F), but not myeloid cells (G). (C) Flow cytometry analysis of the expression of c-Maf and Mrc1 at E16.5 in control and c-Maf^{ΔEC} mice, n=12. (D) Analysis of the medium fluorescence intensity of Mrc1 expression in the ECs in the control and c-Maf^{ΔEC} mice. (E) Flow cytometry analysis of the expression of c-Maf and Fcgr2b at E16.5 in control and c-Maf^{ΔEC} mice. (F) Analysis of the percentage of ECs positive for Fcgr2b in the control and c-Maf^{ΔEC} mice, n≥10. (G) Immunofluorescence analysis of the vascular markers Aqp1, Lyve1, and Emen in control and c-Maf^{ΔEC} mice at E16.5 of development, induced as shown in (D) from n=5 mice

FIGS. 4A-M. c-Maf postnatal deficiency overextends the restricted hematopoietic liver sojourn through an aberrant vascular arterialization. (A) Analysis of the influence of c-Maf on postnatal liver vascular development was performed by 4-hydroxytamoxifen administration from P2 to P4 and at P8 and analyzed at P15 in control and c-Maf^{ΔEC} mice. Flow-assisted cell sorting was performed for the gated CD45⁺, CD3^{neg} and CD45^{neg}CD31⁺ populations, as indicated on the flow chart, from 2 control and 2 c-Maf^{ΔEC} mice. (B) Single cell analysis of the hematopoietic and endothelial cell sorted populations from (A) was performed. UMAP labeling of the different endothelial and hematopoietic populations identified: Endothelial populations: Sinusoids (S 1-2), KO-Sinusoids (KO-S 1-3), Cycling (C), Portal Vein (PV); Hematopoietic populations: Common lymphoid progenitors (CLP), Cycling-CLP (C-CLP), B cells (B 1-3), T cells (T 1-3), Leukocytes (L 1-5) and Kupffer cells; Contaminant cells were identified as: Doublets (D) and Hepatocytes (H). (C) Expression of c-Maf in the endothelial and Kupffer cell clusters of Control and c-Maf^{ΔEC} mice. (D) Proportion analysis shows identification of the generation of a unique cluster of cells within the c-Maf^{ΔEC} mice associated with the c-Maf deficient cells. (E) Flow cytometry analysis of the percentage of Ki67 positive cells within the ECs. T-test analysis *p<0.05 n≥5. (F) Pseudotime analysis of the endothelial cell populations. Arrows show the directionality of changes, associated with the transition between different cells. The top right panel shows the associated pseudotime per sample. (G) Partition-based graph abstraction analysis of transition confidence between endothelial cell clusters in either control or c-Maf^{ΔEC} mice. (H) Volcano plot of the differentially expressed genes between the control and c-Maf^{ΔEC} mice. Several genes associated with the sinusoidal cell and Portal Vein populations are shown. Colors indicate fold change in expression: red-increased and blue-decreased. (I) Immunofluorescence staining of Sca1 and Lyve1 in control and c-Maf^{ΔEC} mice. Representative image of n=5, bar size 100 μm. (J) Quantification by flow cytometry of the expression levels of Fcgr2b in control versus c-Maf^{ΔEC} mice. n≥10. (K) Hematoxylin and cosin staining of control and c-Maf^{ΔEC} livers. Arrows and circles show areas with increase deposition of hematopoietic cells. Representative images of n=5. (L) Percentage of cells in the cluster of Common lymphoid progenitors-CLPs identified from the scRNA-seq in (B). (M) Flow cytometry analysis of CD45⁺ CD45RA⁺ cells positive for CD24a and CD20. T-test analysis *p<0.05 n≥5.

FIGS. 5A-M. c-Maf adult vascular deficiency aberrantly induces a Portal Vein signature of the capillaries and facilitates stress-induced liver fibrosis. (A) Induction of c-Maf deficiency in vascular endothelium was performed by tamoxifen administration after P30 using 3 days on, 3 days off, 3 days on protocol. (B) Analysis of vascular ECs (ECs) show c-Maf deletion within the endothelium, n≥11. (C) Flow cytometry analysis of the percentage of Ki67 positive cells within the ECs. T-test analysis *p<0.05 n=5. (D) RNA-seq analysis of adult liver ECs isolated from control and c-Maf^{ΔEC} mice, showing the differential expression signature between them, n≥11. (E) GSEA analysis of the postnatal sinusoidal gene list from population PS5 identified in FIG. 1C. The analysis shows a decrease in the expression of sinusoidal genes associated within the c-Maf^{ΔEC} mice. (F) GSEA analysis of the Portal Vein gene list from population PV identified in FIG. 1C. The analysis shows an increased in the expression of these genes within the c-Maf^{ΔEC} mice. (G) Flow cytometry quantification of the medium fluorescence intensity of sinusoidal marker Mrc1 expression in control and c-Maf^{ΔEC} mice, n≥11. (H) Flow cytometry quantification of the medium fluorescence intensity of Portal Vein marker Ly6a/Sca1 expression in control and c-Maf^{ΔEC} mice, n≥11. (I-J) Immunofluorescence analysis of the expression of liver vascular markers Mrc1, Ly6a/Sca1, Lyve1, Emen, and Aqp1 in control and c-Maf^{ΔEC} mice. Representative image from n=5, bar size=100 μm. (K) Representative images of Masson's trichrome from n=3 of control and c-Maf^{ΔEC} mice under basal conditions. A small fraction of fibrosis deposition could be observed surrounding the Portal Vein both in control and c-Maf^{ΔEC} mice. (L) Induction of fibrosis using CCl₄ treatment in control and c-Maf^{ΔEC} mice, 1 month after deletion was induced. Representative images of Masson's trichrome from n≥9 of control and c-Maf^{ΔEC} mice, showing the fibrotic deposition in blue. Image shows a zoom region from a tile scan of the whole liver. The white dashed line follows the fibrosis area. Bar size=200 μm. (M) Quantification of the percentage of fibrosis over the total tissue area from n≥9 of control and c-Maf^{ΔEC} mice. Statistical analysis was performed by using a t-test comparing the two groups, with *p<0.05, **p<0.01, ***p<0.001.

FIGS. 6A-F. Human liver EC subpopulations can be identified by the expression of unique differential markers acquired by immunofluorescence and flow cytometry. (A) Human liver CD45^{neg}CD31⁺ cells were sorted and analyzed by single cell RNA-seq analysis (n=1). UMAP analysis of the endothelial cell (EC) compartment from the human liver sorted as CD45^{neg}CD31⁺. Colors represent each population identified based on their differentially expressed markers. (B) Single cell analysis of human liver CD45^{neg}CD31⁺ cells identified for clusters based on the expression of specific markers such as CD31, CDH5, AQP1, STAB2, SELP, or CXCR4. The co-expression of both CD31 and CDH5 were used to identify the ECs. These clusters were associated with the Portal Vein, sinusoids, Central Vein, and hematopoictic plasma cells (CD45^{neg}CD31⁺). (C) Identification of the expression of cluster-associated specific markers between each subpopulation represented as a Heatmap. Colors show the varying expression levels of each gene per cell. (D) Immunofluorescence validation of the vascular markers identified by single cell RNA-seq analysis allows the identification of specific markers associated with the Portal Vein, sinusoids or Central Vein based on the expression of Aqp1, Cd34, Cdh5, Cd14, Lyve1, and Eng. Boxes represent the labeling of the gene in a particular population. Representative images from 3 patients. Bar size=100 μm. (E) Table representing the identification of protein expression by immunofluorescence on the Portal Vein (PV), Sinusoids (Sn) or Central Vein (CV) in the human liver. Green color indicates positive identification of expression in panel (D). (F) Identification by Flow cytometry of the different vascular subpopulations found in the single cell RNA-seq based on the expression of the surface markers CD45, CD31, CD38, CD14, CD34, and CD9, performed in two different human liver samples.

FIGS. 7A-I. c-Maf induces a pro-regenerative liver signature in vascular ECs, enabling long-term sustenance of co-cultured hepatocytes. (A) Expression of c-Maf in the EC subpopulations from the human single cell analysis. UMAP represents the different EC populations identified. The results show an enrichment in the sinusoidal population(S) compared to the Portal Vein (PV) and Central Vein (CV). T-test comparison ***p,0.001. (B) Induction of enhanced expression of c-Maf in human ECs in vitro triggers a morphological change associated with an increase in expression of CD36. (C) Immunofluorescence analysis of the expression of CD31 and CD36 in ECs transduced with a lentivirus control or lentivirus overexpressing c-Maf under the control of doxycycline. Nuclei are stained with Hoechst. Analysis of cell cultures of c-Maf cells, with or without doxycycline, for seven days. Representative image of n=3. Bar size 100 μm. (D) Flow cytometry identification and quantification of induced LSEC (ILSECs) after the administration of doxycycline. Cells overexpressing c-Maf acquire elevated expression of CD26 and CD36. Representative flow of n=3 (different donors). (E) RNA-seq analysis of control and iLSECs cells treated for seven days with doxycycline. Heatmap shows differential expression analysis between the iLSEC population overexpressing c-Maf and control cells from n=3 donor cells. (F) GSEA analysis of the expression values of the human sinusoidal enriched gene list from our single cell analysis identified an upregulation of this signature in the iLSECs from the RNA-seq analysis. (G) Representation of the co-culture of human hepatocytes and iLSECs to generate spheroids. (H) RT-qPCR results indicating relative CYP1A2 expression at day 28 of the co-culture of human hepatocytes with ECs (control or iLSECs). (I) ELISA quantification of the expression of albumin over the 28-day co-culture of hepatocytes with ECs (control or iLSECs). Data is presented as mean±SEM. On panels H and J, t-test analysis was performed comparing control ECs and iLSECs. On panels F and L, one-way ANOVA was performed to compare the different populations. *p<0.05, ***p<0.001, ****p<0.0001.

FIGS. 8A-I. Identification of cell populations across multiple times during liver development using single cell RNA-seq. (A) Liver samples were mechanically homogenized in a solution of Collagenase/Dispase and labeled with CD31 and CD45 antibodies and Dapi to identified live cells. Endothelial cells (ECs) identified as Dapi^{neg}CD45^{neg}CD31⁺ were sorted and single cell RNA-seq analysis was performed using this population. (B) Representation of the gating strategy used to enrich the vascular population prior to performing single cell RNA-seq analysis. The ECs are identified as the red population within the dashed line that is CD45^{neg}CD31⁺ at different fetal and postnatal developmental timepoints. (C, D) UMAP from single cell analysis of sorted liver endothelial cells (ECs; CD45^{neg}CD31⁺) at fetal and postnatal timepoints: E12, E14, E16, E18, P2, P8, P15 and P30. Colors are assigned based on the sample timepoint (C) or cell cluster (D). Labeling corresponds to the different populations identified from the single cell analysis from (D): Cavin3⁺: Cv3, Portal Vein: PV, Central Vein: CV, Fetal sinusoidal EC populations: FS1-5, Postnatal sinusoidal EC populations: PS1-5, Proliferating ECs: P1-3, Cxc110 high expressing ECs (C^{high}): C, Erythro-myeloid progenitors: EMP1-3, Megakaryocytes: M1-2, Lymphocytes: L, Hepatocytes: H, Bile Duct: BD, Kupffer cell Doublets: KD. (E), Identification of specific markers associated to the individual populations identified in the single cell analysis. Colors were assigned as on (D), with the graphical representation of the vasculature including colors used to label each subpopulation. (F) The pan endothelial cell markers Pecam1/Cd31 and Cdh5/VE-cadherin were used to identify vascular populations as double positive cells for these two markers. (G) Identification of vascular fetal enriched markers Pgk1 and Mif, which have elevated expression during early fetal development. (H) During postnatal vascular development the endothelium increases the expression of genes such as Mrc1 and Fcgr2b. (I) Cavin3⁺ population can be identified by the expression of Cavin3. Expression of Cavin3 is restricted in the human adult vasculature to the Hepatic Artery based on the data from the Human Protein Atlas (bar size=25 μm), which were not present together in other large vessels.

FIGS. 9A-D. Immunofluorescence characterization of large vessel development. (A), Violin plots of the expression values of Aqp1, Lyve1 and Emen in the different EC subpopulations. (B), Immunofluorescence analysis of the vascular markers Aqp1, Lyve1 and Emen across fetal and postnatal developmental timepoints. VV/UV refers to the infiltrating vitellin vein and umbilical veins at E12. Aqp1 emergence can be observed within the Portal Vein (PV) at E14 (arrow). Staining of the venous marker endomucin (Emen) can be observed in the Central Vein (CV) from E18 onwards (arrow at E18). Representative images from n=5, Bar size=100 μm. (C) Graphical presentation identifying the acquisition of the different markers in the Portal Vein (PV), Central Vein (CV) and Sinusoids(S). (D) Immunofluorescent analysis of the hepatocyte zonated Portal Vein marker E-cadherin and the centrilobular marker Cyp2E1, with nucleus stained with Dapi. Representative image of n=5. Bar size 100 μm.

FIGS. 10A-U. Single cell pseudotime analysis defines a temporal expression of angiocrine factors in development. (A) Partition-based graph abstraction analysis of transition confidence between developmental stages. (B) Pseudotime prediction of gene expression transition from the EC populations. (C) UMAP representation of CD34 expression in the EC clusters. Higher expression levels were observed at E12. (D-E) Immunofluorescence analysis of Lyve1 and CD34 at E12 (D) and E14 (E) of fetal development. CD34 stained was present in all ECs at E12 whereas restricted to the Portal Vein at E14. “PV” represents portal vein and “CV” represents central vein. Bar size 100 μm. (F-U) Expression of genes identified by pseudotime analysis that has significant expression changes from E12 of fetal developmental timepoints to postnatal day P30. Expression of Cdk1, lgf2r, Mest, Lyve1, Cdh4, and Plvap (F-K) were more enriched during fetal development, whereas Vwf, Stab2, Adam23, Ltbp4, Il6st, Dpp4, Ptprb, Bmp2, Wnt2, H2-K1 (L-U) were more enriched postnatally.

FIGS. 11A-N. c-Maf is enriched in the liver sinusoidal cell population and sustained over time. (A) Expression levels of the Maf family members in liver ECs from the Tabula Muris database. (B) UMAP expression of the transcription factors Gata4 and c-Maf within the present study's single cell RNAseq analysis. (C) Expression levels represented by violin plots of Gata4 and c-Maf were assessed in all EC populations identified in the single cell RNA-seq analysis. (D) Quantitative Real Time-PCR (qRT-PCR) measure of c-Maf expression levels in mouse liver ECs cultured for 48 hours in the presence of Bmp9, Lif, Il6, or a negative control (n=3). (E) Expression of liver-enriched genes measured by qRT-PCR of liver ECs cultured for 48 hours in the presence of Bmp9. Student's t-test *p<0.05 (n=3). (F) Flow cytometry identification of endothelial cells as CD45^{neg}CD31⁺ across fetal E12, E14, E16, E18 and 4 weeks mice. (G-J) Flow cytometry analysis of the expression levels of c-Maf and Mrc1 (G, H), and c-Maf and Fcgr2b (I, J) in the liver ECs. Colors indicate the quadrant from the flow cytometry gating. Percentage of cells identified by flow cytometry that were double positive for c-Maf and Mrc1 (H) or Fcgr2b (J) across different developmental timepoints. Student's t-test comparing E12 and each other timepoint was performed ***p<0.001 (n≥5). (K) Flow cytometry analysis of the endothelial cells CD45^{neg}CD31⁺ and myeloid CD45⁺ CD68⁺ compartment in mice at E16 from Control and c-Maf^{ΔEC} mice. Labels indicate identification of the different populations. (L) Expression of c-Maf was not affected in myeloid cells after tamoxifen administration at E16. Student's t-test p>0.05 (n=12). (M) Percentage of cells positive for Ki67 analyzed by flow cytometry at E16. Student's t-test p>0.05 (n≥9). (N) Analysis of the change in fractal dimension of the vascular network in control and c-Maf^{ΔEC} mice at E16. Student's t-test **p<0.01 (n≥9).

FIGS. 12A-H. c-Maf deficient ECs differentially segregate postnatally. (A) Single cell RNA-seq was performed at P15 for one male and one female mouse each from the two groups (Control and c-Maf^{ΔEC}). Samples from each group were sorted and combined for the single cell analysis. Subsequently the two samples were bioinformatically split into their original four samples based on the expression of sexually dimorphic genes in each cell. All four samples were plotted in a UMAP showing the overlap of all samples. (B) Identification of individual populations from the scRNA-seq analysis based on expression of specific markers. Endothelial populations: Sinusoids (S1, S2), KO-Sinusoids (KO-S 1-3), Cycling (C), Portal Vein (PV); Hematopoictic populations: Common lymphoid progenitors (CLP), Cycling-CLP (CLP-C), B cells (B 1-3), T cells (T 1-3), Leukocytes (L 1-5) and Kupffer cells (K); Contaminant cells were identified as: Doublets (D) and Hepatocytes (H). (C) UMAP representation of the expression levels of c-Maf in control and c-Maf^{ΔEC} mice in all populations. (D) Percentage of cells positive for c-Maf in the ECs and Kupffer cell populations shows no significant changes in the Kupffer cell population but significant decreased in the EC clusters of c-Maf^{ΔEC} mice. Fisher test ***p<0.001. (E) Flow cytometry quantification of the percentage of ECs expressing c-Maf in control and c-Maf^{ΔEC} mice. Student's t-test ***p<0.001 (n≥10). (F) Pseudotime changes in the control and c-Maf^{ΔEC} mice identifying a shorter transitional stage for the c-Maf^{ΔEC} mice. Student's t-test ***p<0.001. (G) Flow cytometry quantification of the medium fluorescent intensity of Mrc1 and Fegr2b expression levels in control and c-Maf^{ΔEC} mice. Student's t-test p<0.001 (n≥10). (H) CFU analysis of sorted CD45 total cells from control and c-Maf^{ΔEC} mice. Student's t-test **p<0.01 (n=5).

FIGS. 13A-L. Adult vascular c-Maf deficiency is associated with a loss of the sinusoidal program. (A-B) Analysis of c-Maf expression in macrophages CD45⁺ CD68⁺ (A) and Kupffer cells CD45⁺ CD68⁺F4/80⁺ CDH5⁺ (B) shows no significant differences. Student's t-test ***p>0.05 (n≥11 (A), and n=5 (B). (C) GSEA analysis of the expression levels of postnatal sinusoidal genes with an associated c-Maf motif. (D-E) Flow cytometry quantification of the medium fluorescent intensity in the endothelial cell population of the sinusoidal marker Fcgr2b (D) and the arterial marker Cd9 (E). Student's t-test ***p<0.001 (n≥11). (F) Quantification of the vascular network interactome assay as the fractal dimension of the vasculature in control and c-Maf^{ΔEC} mice. Student's t-test p>0.05 (n=5). (G) Electron microscopy of livers from Control and c-Maf^{ΔEC} mice from n=3 showing presence of fenestration in c-Maf^{ΔEC} mice. Arrows indicate fenestrations. Bar size 1 μm. (H) Immunofluorescence analysis of the portal to centrilobular makers E-cadherin and Cyp2E1 respectively shows no changes in the overall zonation between control and c-Maf^{ΔEC} mice from n=5. Bar size 100 μm. (I) Immunofluorescence quantification analysis of the area positive for GS staining in Control and C-Maf^{ΔEC} mice. Circular areas indicate glutamine synthetase (GS) positive staining in hepatocytes. Student's t-test **p<0.01 (n=5). Bar size 100 μm. (J) Immunofluorescence quantification analysis of clonal vascular size of Confetti mice crossed with c-Maf^{ΔEC} mice, compared to control adult mice. Circular areas indicate representative clones expanding in the c-Maf^{ΔEC} mice. “V” represents veins. Student's t-test **p<0.01 (n≥6). Bar size 100 μm. (K) Flow cytometry analysis of the expression levels of c-Maf and Mrc1 in mice treated biweekly with oil and 25% CCl4 diluted in oil for one month. Data represents the percentage of cells that were double negative for Mrc1 and c-Maf in both groups. Student's t-test *p<0.05 (n=5). (L) Quantification of the percentage of fibrosis over the total tissue area of control and c-Maf^{ΔEC} mice in basal conditions. Student's t-test ***p>0.05 (n=3).

FIGS. 14A-I. Enhanced expression of c-Maf in endothelial cells (ILSECs) induces the expression of surface markers associated to human liver sinusoidal cells. (A) Volcano plot comparing the Portal Vein and Central Vein gene signatures. (B) Immunofluorescent analysis of a 17-week human liver sample label with Aqp1, Cd34, and Lyve1 allows the identification of the portal vein (PV), sinusoids(S), and central vein (CV). Boxes represent the labeling of the gene at a particular population. Bar size=50 μm. (C) Flow cytometry expression analysis of vascular markers in a 17-week human liver sample shows the expression of CD14 and CD34 by liver ECs at this fetal developmental stage. At 17 weeks of human fetal development, the population of CD45^{neg}CD31⁺ CD38⁺ plasma cells could not be observed. (D) ECs transduced with a lentiviral control vector or c-Maf inducible vector co-cultured with or without doxycycline for seven days. Representative image of n=3. Bar size=250 μm. (E) Representative image of human liver EC culture in vitro after isolation with CD144 (VEcadherin (CDH5)) beads, n=1. Bar size=250 μm. (F-G) Quantification of CD14 (F) and MRC1 (G) expression levels by flow cytometry of iLSECs compared to negative (non-reprogrammed) cells and cells cultured without doxycycline. Student's t-test *p<0.05, ***p<0.001 (n=3 different donors). (H) Identification of ILSECs as CD36⁺ CD14⁺ by flow cytometry. Representative image of n=3. Expression of c-Maf in ILSECs compared to negative (CD36^{neg}CD14^{neg}) cells or cells cultured without doxycycline. One way ANOVA **p<0.01, ***p<0.001 (n=3 different donors). Histogram represents expression levels of c-Maf. (I) Electron microscopy analysis of the co-culture of hepatocytes (H) with control ECs or iLSECs. Arrows indicate the existence of empty cavities in the iLSECs, but not the controls, that emulate fenestrations. White bar represents 1 μm.

### DETAILED DESCRIPTION

It has been recognized herein that the enhanced expression of the transcription factor MAF (c-Maf) in endothelial cells promotes the generation of induced liver sinusoidal endothelial cells (ILSECs). ILSECs obtained as a result of enhanced expression of c-Maf and selection for at least two of the cell surface markers: CD26, CD30 and CD14, allow the co-culture with hepatocytes for long periods of time (28 days or longer). The use of these iLSECs has great potential on the development of new approaches to test drugs on hepatocytes or ES/iPSC derived hepatocytes. These cells can also be used in regenerative medicine to treat liver disorders by inoculating them into the body, or by using them in bioengineering livers to be transplanted.

### Endothelial Cells (ECs)

Endothelial cells form a single cell layer that lines all blood vessels and regulates exchanges between the bloodstream and the surrounding tissues. Signals from endothelial cells organize the growth and development of connective tissue cells that form the surrounding layers of the blood-vessel wall. New blood vessels can develop from the walls of existing small vessels by the outgrowth of endothelial cells, which have the capacity to form hollow capillary tubes even when isolated in culture. Endothelial cells of developing arteries and veins express different cell-surface proteins, which may control the way in which they link up to create a capillary bed.

Endothelial cells suitable for use herein include endothelial cells without a liver signature. By “liver signature” is meant that expression of cell surface markers from the human liver sinusoids including, e.g., CD14 and MRC1; preferably including at least two of CD14, CD26 and CD36, as well as MRC1. Examples of endothelial cells suitable for use herein are human umbilical vein endothelial cells (HUVECs), adipose-derived endothelial cells, organ-specific endothelial cells, endothelial stem cells, and endothelial progenitor cells. Examples of the organ-specific endothelial cells are heart-specific endothelial cells, muscle-specific endothelial cells, kidney-specific endothelial cells, testis-specific endothelial cells, ovary-specific endothelial cells, lymphoid-specific endothelial cells, pancreas-specific endothelial cells, brain-specific endothelial cells, lung-specific endothelial cells, bone marrow-specific endothelial cells, spleen-specific endothelial cells, large intestine-specific endothelial cells, small intestine-specific endothelial cells, and ovary or testicular endothelial cells. Also suitable for use herein are endothelial cells which have been isolated from the liver as liver-specific endothelial cells and cultured in vitro after the isolation. Liver-specific endothelial cells may lose their liver signature as a result of an in vitro culture and can be converted to iLSECs by enhanced expression of c-Maf.

### Human Umbilical Vein Endothelial Cells (HUVECs)

Human umbilical vein endothelial cells (HUVECs) are primary cells isolated from the vein of the umbilical cord. HUVECs have been used for studying endothelial cell function, with applications including hypoxia, inflammation, oxidative stress, response to infection, and both normal and tumor-associated angiogenesis. HUVECs exhibit a characteristic “cobblestone” morphology and express the endothelial-specific marker proteins platelet endothelial cell adhesion molecule (PECAM-1/CD31), vascular endothelial (VE)-cadherin, and von Willebrand factor (VWF).

### Liver Sinusoidal Endothelial Cells (LSECs)

Liver sinusoidal endothelial cells (LSECs) are highly specialized endothelial cells representing the interface between blood cells on the one side and hepatocytes and hepatic stellate cells on the other side. LSECs represent a permeable barrier. In physiological conditions, LSECs regulate hepatic vascular tone contributing to the maintenance of a low portal pressure despite the major changes in hepatic blood flow occurring during digestion. LSECs maintain hepatic stellate cell quiescence, thus inhibiting intrahepatic vasoconstriction and fibrosis development. In pathological conditions, LSECs play a key role in the initiation and progression of chronic liver diseases. Indeed, they become capillarized and lose their protective properties, and they promote angiogenesis and vasoconstriction. LSECs are implicated in liver regeneration following acute liver injury or partial hepatectomy since they renew from LSECs and/or LSEC progenitors, they sense changes in shear stress resulting from surgery, and they interact with platelets and inflammatory cells. LSECs also play a role in hepatocellular carcinoma development and progression, in ageing, and in liver lesions related to inflammation and infection.

iLSECs

Endothelial cells including endothelial cells without a liver signature can be converted to liver sinusoidal endothelial cells (LSECs) by enhanced expression of specific transcription factors. The LSECs, generated thereby, are termed as induced liver sinusoidal endothelial cells (ILSECs). In the present disclosure, enhanced expression of a specific transcription factor, c-Maf, induces sinusoidal identity in the generic human endothelial cells (such as HUVECs) and generates iLSECs. Thus, c-Maf represents an inducible specification factor for liver sinusoids and sets the stage to devise future approaches for therapeutic liver repair.

In some embodiments, iLSECs provided herein express at least 2 of the cell surface markers CD26, CD36, and CD14. In some embodiments, ILSECs provided herein express all 3 of the cell surface markers CD26, CD36, and CD14. In some embodiments, ILSECs provided herein also express MRC1. In some embodiments, ILSECs provided herein also express CD26, CD36, CD14, and MRC1 markers at the cell surface. In some embodiments, ILSECs provided herein additionally express VE-Cadherin.

iLSECs display a cobblestone-like morphology and contain many fenestrae (small pores) with uniform diameters of 100-150 nm, thus creating open channels for the exchange of substances between the blood and liver parenchyma.

Like LSECs, the iLSECs can regulate hepatic vascular tone contributing to the maintenance of a low portal pressure despite the major changes in hepatic blood flow occurring during digestion. ILSECs are capable of maintaining hepatic stellate cell quiescence, thus inhibiting intrahepatic vasoconstriction and fibrosis development.

The iLSECs provided herein are shown to have the ability to support a co-culture with hepatocytes and maintain hepatocyte cultures for a longer period of time compared to hepatocyte cultures without the iLSECs. For example, the iLSECs provided herein can maintain hepatocytes (and their functionality) in a coculture for at least 20 days to at least 30 days, e.g., at least 28 days. The long-term sustainability of hepatocyte function can be assessed by CYP1A2 (a key enzyme in liver metabolism) expression and maintenance of albumin production.

### Transcription Factors

A transcription factor (TF) (or sequence-specific DNA-binding factor) is a protein that controls the rate of transcription of genetic information from DNA to messenger RNA, by binding to a specific DNA sequence. The function of TFs is to regulate (turn on and off) genes in order to make sure that they are expressed in the desired cells at the right time and in the right amount throughout the life of the cell and the organism. Groups of TFs function in a coordinated fashion to direct cell division, cell growth, and cell death throughout life; cell migration and organization during embryonic development; and intermittently in response to signals from outside the cell, such as a hormone. There are 1500-1600 TFs in the human genome.

### MAF Transcription Factor

The Maf transcription factor family is composed of 7 members divided into two subclasses: the large Maf proteins composed of MAFA/L-MAF, MAFB, MAF/c-Maf, and NLR (neural retina leucine zipper), and the small Maf proteins, MAFK, MAFG, and MAFF, which lack the amino-terminal transactivation domain. Maf transcription factors form a distinct subfamily of the basic leucine zipper (bZip) transcription factors. Maf genes have been identified in a wide range of higher eukaryotes, including both vertebrate and invertebrate species. These proteins are unique among the bZip factors in that they contain a highly conserved extended homology region (EHR), or ancillary DNA binding region, in addition to a typical basic region, and both regions are involved in target DNA sequence recognition. Maf transcription factors regulate tissue-specific gene expression and cell-differentiation in a wide variety of tissues and are also involved in human diseases and oncogenic transformation. Tissue-specific expression involves Maf binding to Maf-recognition elements (MAREs) in the regulatory regions of target genes and interacting with other transcription factors.

c-Maf

c-Maf is a member of the basic leucine zipper transcription factors belonging to the AP-1 family. c-Maf protein, a member of the transcription factor class of proteins that control large sets of genes, is required for naive endothelial cells to mature specifically into liver sinusoidal blood vessels.

It has been demonstrated in this disclosure that the peri-natal induction of the transcription factor c-Maf is a critical switch for the sinusoidal identity determination. It has been demonstrated herein that endothelium-restricted deletion of c-Maf disrupts liver sinusoidal development, aberrantly expands postnatal liver hematopoiesis, promotes excessive postnatal sinusoidal proliferation, and aggravates liver pro-fibrotic sensitivity to chemical insult. Further, it has been demonstrated herein that enforced c-Maf enhanced expression in generic human endothelial cells switches on a liver sinusoidal transcriptional program that maintains hepatocyte function. Thus, this disclosure provides the recognition that c-Maf represents an inducible intra-organotypic and niche-responsive molecular determinant of hepatic sinusoidal cell identity and lays the foundation for the strategies for vasculature-driven liver repair.

In some embodiments, exemplary c-Maf sequences suitable for use herein include the following:
SEQ ID NO: 1-Human c-Maf DNA sequence,
SEQ ID NO: 2-Human c-Maf protein sequence,
SEQ ID NO: 3-Human c-Maf isoform DNA sequence,
SEQ ID NO: 4-Human c-Maf isoform protein sequence,
SEQ ID NO: 5-Human c-Maf isoform DNA sequence,
SEQ ID NO: 6-Human c-Maf isoform protein sequence,
SEQ ID NO: 7-Mouse c-Maf DNA sequence,
SEQ ID NO: 8-Mouse c-Maf protein sequence,
SEQ ID NO: 9-Mouse c-Maf isoform DNA sequence,
SEQ ID NO: 10-Mouse c-Maf isoform protein sequence.

Also suitable for use herein are nucleic acid molecules encoding another naturally occurring c-Maf protein isoform not specifically listed above, nucleic acid molecules encoding a polypeptide having at least 85%, 90%, 95%, 98%, 99% or greater sequence identity to a naturally occurring c-Maf protein (such as a human or mouse c-Maf protein listed herein above) and having the same biological activities of a naturally occurring c-Maf protein, as well as nucleic acid molecules encoding a polypeptide that differs from a naturally occurring c-Maf protein (such as a human or mouse c-Maf protein listed herein above) by not more than 3 amino acids, not more than 2 amino acids, or not more than 1 amino acid and having the same biological activities of a naturally occurring c-Maf protein.

Enhanced Expression of c-Maf

Enhanced expression of a molecule is defined as up-regulated expression of that molecule in a host cell compared to its endogenous or basal level expression. In some embodiments, the enhanced expression of c-Maf protein results in 20%, 30%, 40%, 50%, 60%, 70%, 80%, 90% or 100% higher than the endogenous or basal level expression of c-Maf protein. In some embodiments, the enhanced expression of c-Maf protein can be 2 fold, 5 fold, 10 fold, 20 fold, 30 fold, 40 fold, 50 fold, 60 fold, 70 fold, 80 fold, 90 fold or 100 fold higher than the endogenous or basal level expression of c-Maf protein. The enhanced expression of a c-Maf protein leads to an elevated level of the c-Maf protein in the host cell. In some embodiments, the enhanced expression of a c-Maf protein results in 20%, 30%, 40%, 50%, 60%, 70%, 80%, 90% or 100% higher in the protein level than the endogenous or basal protein level of c-Maf protein.

In some embodiments, enhanced expression of c-Maf protein can be achieved by introducing to a population of endothelial cells (particularly endothelial cells without a liver signature e.g., human generic endothelial cells such as HUVECs) an exogenous nucleic acid encoding a c-Maf protein. In some embodiments, the exogenous nucleic acid comprises a c-Maf nucleic acid operably linked to a promoter capable of expressing c-Maf in the recipient endothelial cells. In some embodiments, the exogenous nucleic acid is a viral vector. In some embodiments, the exogenous nucleic acid is modified mRNA encoding a c-Maf protein. In some embodiments, the exogenous nucleic acid is introduced into the host cell using a dead CrisprCas9-VP16 or any other dead CrisprCas9 system, which system facilitates the integration of the exogenous nucleic acid into the host genome. In some embodiments, upregulation of c-Maf such as an endogenous c-Maf is achieved using cytokines. In some embodiments, upregulation of c-Maf such as an endogenous c-Maf is achieved using chemicals such as small molecule compounds.

### Exogenous Nucleic Acids

Exogenous nucleic acids are nucleic acids originating outside an organism that has been introduced into the organism. Exogenous nucleic acids may enter the nucleus, where some are absorbed and/or blocked by heterochromatin and others integrate into chromosomes. Examples of exogenous nucleic acids suitable for use to achieve enhanced expression of c-Maf include nucleic acid vectors such as plasmids or viral vectors, or mRNAs including modified mRNAs.

Modified RNA (modRNA)

A nucleoside is a molecule including a nitrogenous base (i.e., a nucleobase) linked to a pentose (e.g., deoxyribose or ribose) sugar. Nitrogenous bases which form nucleosides include adenine, guanine, cytosine, 5-methyl cytosine, uracil, and thymine. Suitable ribonucleosides (which comprise ribose as the pentose sugar) include, e.g., adenosine (A), guanosine (G), 5-methyluridine (m5U), uridine (U), and cytidine (C). Nucleotides are molecules including a nucleoside (e.g., a ribonucleoside) and a phosphate group. Ribonucleotides include, e.g., adenosine monophosphate, adenosine diphosphate, adenosine triphosphate, guanosine monophosphate, guanosine diphosphate, guanosine triphosphate, cytidine monophosphate, cytidine diphosphate, cytidine triphosphate, uridine monophosphate, uridine diphosphate, uridine triphosphate, and derivatives thereof.

Modified RNA, or modRNA, is a synthetic modified RNA that can be used for expression of a gene of interest. Chemical modifications to a ribonucleotide included in modRNA may stabilize an RNA molecule, blunt an immune response, or enhance transcription. Additionally, unlike delivery of protein agents directly to a cell, which can activate the immune system, the delivery of modRNA can be achieved without immune impact. For example, substitution of uridine and cytidine with pseudouridine or N1-methylpseudouridine and 5-methylcytidine, respectively, drastically reduces the immune response elicited from exogenous RNA without such substitutions. Stability and translational efficiency from an RNA molecule may also be increased by including a 3′-O-Me-m7G (5′) ppp (5′) G Anti Reverse Cap Analog (ARCA) at the 5′ end of the RNA molecule.

modRNA may encompass an RNA molecule with at least uridine substituted with pseudouridine. modRNA may encompass an RNA molecule with at least cytidine substituted with 5-methylcytidine. modRNA may encompass an RNA molecule including the modified nucleoside 5-methylcytidine (5mC). modRNA may encompass an RNA molecule including the modified nucleoside 2-Thiouridine-5′-Triphosphate (2-thio ψU). modRNA may encompass an RNA molecule with at least the modified nucleoside 1-Methylpseudouridine-5′-Triphosphate (1-mψU). modRNA may encompass an RNA molecule with at least the modified nucleoside N1-methyl-pseudouridine (N1mΨ) substituted for uridine. modRNA may encompass an RNA molecule wherein at least 5′ triphosphates are removed. modRNA may encompass an RNA molecule wherein at least a 3′-O-Me-m7G (5′) ppp (5′) G Anti Reverse Cap Analog (ARCA) cap or C₃₂H₄₃N₁₅O₂₄P₄ CleanCap Reagent AG is included in a 5′ untranslated regions of the RNA molecule.

modRNAs may be prepared by in vitro transcription. modRNA may be in vitro transcribed, e.g., from a linear DNA template using one or more reagents selected from a cap analog, guanosine triphosphate, adenosine triphosphate, cytidine triphosphate, uridine triphosphate, and derivatives thereof. A cap analog may be selected from Anti-Reverse Cap Analog (ARCA) 3′-O-Me-m7G (5′) ppp (5′) G, standard cap analog m7G (5′) ppp (5′) G, unmethylated cap analog G (5′) ppp (5′) G, methylated cap analog for A+1 sites m7G (5′) ppp (5′) A, and unmethylated cap analog for A+1 sites G (5′) ppp (5′) A. In certain examples, a cap analog is Anti-Reverse Cap Analog (ARCA) 3′-O-Me-m7G (5′) ppp (5′) G. According to some examples, modRNA may be in vitro transcribed from a plasmid template using one or more reagents selected from 3′-O-Me-m7G (5′) ppp (5′) G, guanosine triphosphate, adenosine triphosphate, cytidine triphosphate, N1-methylpseudouridine-5-triphosphate, and any one or more of the aforementioned examples of modRNA, or others, without limitation and in any combination. Additional suitable modifications to a modRNA or mRNA molecule are well known in the art (e.g., U.S. Pat. No. 8,278,036 to Kariko et al.; U.S. Pat. No. 10,086,043 to Chien et al.; U.S. Patent Application Publication No. 2019/0203226 to Zangi et al.; and U.S. Patent Application Publication No. 2018/0353618 to Burkhardt et al.; all of which are hereby incorporated by reference in their entirety). In some embodiments, the nucleoside that is modified in the modRNA is a uridine (U), a cytidine (C), an adenine (A), or guanine (G). The modified nucleoside can be, for example, m5C (5-methylcytidine), m6A (N⁶-methyladenosine), s2U (2-thiouridien), ψ (pseudouridine), or Um (2-O-methyluridine). Some exemplary chemical modifications of nucleosides in the modRNA molecule may further include, for example and without limitation, pyridine-4-one ribonucleoside, 5-aza-uridine, 2-thio-5-aza uridine, 2-thiouridine, 4-thio pseudouridine, 2-thio pseudouridine, 5-hydroxyuridine, 3-methyluridine, 5-carboxymethyl uridine, 1-carboxymethyl pseudouridine, 5-propynyl uridine, 1-propynyl pseudouridine, 5-taurinomethyluridine, 1-taurinomethyl pseudouridine, 5-taurinomethyl-2-thio uridine, 1-taurinomethyl-4-thio uridine, 5-methyl uridine, 1-methyl pseudouridine, 4-thio-1-methyl pseudouridine, 2-thio-1-methyl pseudouridine, 1-methyl-1-deaza pseudouridine, 2-thio-1-methyl-1-deaza pseudouridine, dihydrouridine, dihydropseudouridine, 2-thio dihydrouridine, 2-thio dihydropseudouridine, 2-methoxyuridine, 2-methoxy-4-thio uridine, 4-methoxy pseudouridine, 4-methoxy-2-thio pseudouridine, 5-aza cytidine, pseudoisocytidine, 3-methyl cytidine, N4-acetylcytidine, 5-formylcytidine, N4-methylcytidine, 5-hydroxymethylcytidine, 1-methyl pseudoisocytidine, pyrrolo-cytidine, pyrrolo-pseudoisocytidine, 2-thio cytidine, 2-thio-5-methyl cytidine, 4-thio pseudoisocytidine, 4-thio-1-methyl pseudoisocytidine, 4-thio-1-methyl-1-deaza pseudoisocytidine, 1-methyl-1-deaza pseudoisocytidine, zebularine, 5-aza zebularine, 5-methyl zebularine, 5-aza-2-thio zebularine, 2-thio zebularine, 2-methoxy cytidine, 2-methoxy-5-methyl cytidine, 4-methoxy pseudoisocytidine, 4-methoxy-1-methyl pseudoisocytidine, 2-aminopurine, 2,6-diaminopurine, 7-deaza adenine, 7-deaza-8-aza adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N⁶-methyladenosine, N⁶-isopentenyladenosine, N⁶-(cis-hydroxyisopentenyl) adenosine, 2-methylthio-N⁶-(cis-hydroxyisopentenyl) adenosine, N⁶-glycinylcarbamoyladenosine, N⁶-threonylcarbamoyladenosine, 2-methylthio-N⁶-threonyl carbamoyladenosine, N⁶,N⁶-dimethyladenosine, 7-methyladenine, 2-methylthio adenine, 2-methoxy adenine, inosine, 1-methyl inosine, wyosine, wybutosine, 7-deaza guanosine, 7-deaza-8-aza guanosine, 6-thio guanosine, 6-thio-7-deaza guanosine, 6-thio-7-deaza-8-aza guanosine, 7-methyl guanosine, 6-thio-7-methyl guanosine, 7-methylinosine, 6-methoxy guanosine, 1-methylguanosine, N²-methylguanosine, N²,N²-dimethylguanosine, 8-oxo guanosine, 7-methyl-8-oxo guanosine, 1-methyl-6-thio guanosine, N²-methyl-6-thio guanosine, or N²,N²-dimethyl-6-thio guanosine.

In some embodiments, modifications made to the modRNA are independently selected from 5-methylcytosine, pseudouridine, and 1-methylpseudouridine.

In some embodiments, the modRNA comprises a modified uracil selected from the group consisting of pseudouridine (ψ), pyridine-4-one ribonucleoside, 5-aza uridine, 6-aza uridine, 2-thio-5-aza uridine, 2-thio uridine (s2U), 4-thio uridine (s4U), 4-thio pseudouridine, 2-thio pseudouridine, 5-hydroxy uridine (ho⁵U), 5-aminoallyl uridine, 5-halo uridine (e.g., 5-iodom uridine or 5-bromo uridine), 3-methyl uridine (m³U), 5-methoxy uridine (mo⁵U), uridine 5-oxyacetic acid (cmo⁵U), uridine 5-oxyacetic acid methyl ester (mcmo⁵U), 5-carboxymethyl uridine (cm⁵U), 1-carboxymethyl pseudouridine, 5-carboxyhydroxymethyl uridine (chm⁵U), 5-carboxyhydroxymethyl uridine methyl ester (mchm⁵U) , 5-methoxycarbonylmethyl uridine (mcm⁵U), 5-methoxycarbonylmethyl-2-thio uridine (mcm⁵s2U), 5-aminomethyl-2-thio uridine (nm⁵s2U), 5-methylaminomethyl uridine (mnm⁵U), 5-methylaminomethyl-2-thio uridine (mnm5s2U), 5-methylaminomethyl-2-seleno uridine (mnm⁵se2U), 5-carbamoylmethyl uridine (ncm⁵U), 5-carboxymethylaminomethyl uridine (cmnm⁵U), 5-carboxymethylaminomethyl-2-thio uridine (cmnm⁵s2U), 5-propynyl uridine, 1-propynyl pseudouridine, 5-taurinomethyl uridine (τcm⁵U), 1-taurinomethyl pseudouridine, 5-taurinomethyl-2-thio uridine (^{TM5}s2U), 1-taurinomethyl-4-thio pseudouridine, 5-methyl uridine (m⁵U, e.g., having the nucleobase deoxythymine), 1-methyl pseudouridine (m¹ψ), 5-methyl-2-thio uridine (m⁵s2U), 1-methyl-4-thio pseudouridine (misty), 4-thio-1-methyl pseudouridine, 3-methyl pseudouridine (m³ψ), 2-thio-1-methyl pseudouridine, 1-methyl-1-deaza pseudouridine, 2-thio-1-methyl-1-deaza pseudouridine, dihydrouridine (D), dihydropseudouridine, 5,6-dihydrouridine, 5-methyl dihydrouridine (m⁵D), 2-thio dihydrouridine, 2-thio dihydropseudouridine, 2-methoxy uridine, 2-methoxy-4-thio uridine, 4-methoxy pseudouridine, 4-methoxy-2-thio pseudouridine, N¹-methyl pseudouridine, 3-(3-amino-3-carboxypropyl) uridine (acp3U), 1-methyl-3-(3-amino-3-carboxypropyl) pseudouridine (acp³ψ), 5-(isopentenylaminomethyl) uridine (inm⁵U), 5-(isopentenylaminomethyl)-2-thio uridine (inm⁵s2U), α-thio uridine, 2′-O-methyl uridine (Um), 5,2′-O-dimethyl uridine (m⁵Um), 2′-O-methyl pseudouridine (ψm), 2-thio-2′-O-methyl uridine (s2Um), 5-methoxycarbonylmethyl-2′-O-methyl uridine (mcm⁵Um), 5-carbamoylmethyl-2′-O-methyl uridine (ncm⁵Um), 5-carboxymethylaminomethyl-2′-O-methyl uridine (cmnm⁵Um), 3,2′-O-dimethyl uridine (m³Um), 5-(isopentenylaminomethyl)-2′-O-methyl uridine (inm⁵Um), 1-thio uridine, deoxythymidine, 2′-F-ara uridine, 2′-F uridine, 2′-OH-ara uridine, 5-(2-carbomethoxyvinyl) uridine, and 5-3-(1-E-propenylamino) uridine.

In some embodiments, the modRNA comprises a modified cytosine selected from the group consisting of 5-aza cytidine, 6-aza cytidine, pseudoisocytidine, 3-methyl cytidine (m³C), N⁴-acetyl cytidine (act), 5-formyl cytidine (f⁵C), N⁴-methyl cytidine (m⁴C), 5-methyl cytidine (m⁵C), 5-halo cytidine (e.g., 5-iodo cytidine), 5-hydroxymethyl cytidine (hm5C), 1-methyl pseudoisocytidine, pyrrolo-cytidine, pyrrolo-pseudoisocytidine, 2-thio cytidine (s²C), 2-thio-5-methyl cytidine, 4-thio pseudoisocytidine, 4-thio-1-methyl pseudoisocytidine, 4-thio-1-methyl-1-deaza pseudoisocytidine, 1-methyl-1-deaza pseudoisocytidine, zebularine, 5-aza zebularine, 5-methyl zebularine, 5-aza-2-thio zebularine, 2-thio zebularine, 2-methoxy cytidine, 2-methoxy-5-methyl cytidine, 4-methoxy pseudoisocytidine, 4-methoxy-1-methyl pseudoisocytidine, lysidine (k2C), alpha-thio cytidine, 2′-O-methyl cytidine (Cm), 5,2′-O-dimethyl cytidine (m⁵Cm), N⁴-acetyl-2′-O-methyl cytidine (ac⁴Cm), N⁴,2′-O-dimethyl cytidine (m⁴Cm), 5-formyl-2′-O-methyl cytidine (f⁵Cm), N⁴,N⁴,2′-O-trimethyl cytidine (m⁴₂Cm), 1-thio cytidine, 2′-F-ara cytidine, 2′-F cytidine, and 2′-OH-ara cytidine.

In some embodiments, the modRNA comprises a modified adenine selected from the group consisting of 2-amino purine, 2,6-diamino purine, 2-amino-6-halo purine (e.g., 2-amino-6-chloro purine), 6-halo purine (e.g., 6-chloro purine), 2-amino-6-methyl purine, 8-azido adenosine, 7-deaza adenine, 7-deaza-8-aza adenine, 7-deaza-2-amino purine, 7-deaza-8-aza-2-amino purine, 7-deaza-2,6-diamino purine, 7-deaza-8-aza-2,6-diamino purine, 1-methyl adenosine (m¹A), 2-methyl adenine (m²A), N⁶-methyl adenosine (m⁶A), 2-methylthio-N⁶-methyl adenosine (ms²m⁶A), N⁶-isopentenyl adenosine (i⁶A), 2-methylthio-N⁶-isopentenyl adenosine (ms²i⁶A), N⁶-(cis-hydroxyisopentenyl) adenosine (io⁶A), 2-methylthio-N⁶-(cis-hydroxyisopentenyl) adenosine (ms²io⁶A), N⁶-glycinylcarbamoyl adenosine (g⁶A), N⁶-threonylcarbamoyl adenosine (t⁶A), N⁶-methyl-N⁶-threonylcarbamoyl adenosine (m⁶t⁶A), 2-methylthio-N⁶-threonylcarbamoyl adenosine (ms²g⁶A), N⁶,N⁶-dimethyl adenosine (m⁶₂A), N⁶-hydroxynorvalyIcarbamoyl adenosine (hn⁶A), 2-methylthio-N⁶-hydroxynorvalylcarbamoyl adenosine (ms²hn⁶A), N⁶-acetyl adenosine (ac⁶A), 7-methyl adenine, 2-methylthio adenine, 2-methoxy adenine, alpha-thio adenosine, 2′-O-methyl adenosine (Am), N⁶,2′-O-dimethyl adenosine (m⁶Am) N⁶,N⁶,2′-O-trimethyl adenosine (m⁶₂Am), 1,2′-O-dimethyl adenosine (m¹Am), 2′-O-ribosyl adenosine (phosphate) (Ar(p)), 2-amino-N⁶-methyl purine, 1-thio adenosine, 8-azido adenosine, 2′-F-ara adenosine, 2′-F adenosine, 2′-OH-ara adenosine, and N⁶-(19-amino-pentaoxanonadecyl) adenosine.

In some embodiments, the modRNA comprises a modified guanine selected from the group consisting of inosine (I), 1-methyl inosine (m¹I), wyosine (imG), methylwyosine (mimG), 4-demethyl wyosine (imG-14), isowyosine (imG2), wybutosine (yW), peroxywybutosine (o₂yW), hydroxywybutosine (OHyW), undermodified hydroxywybutosine (OHyWy), 7-decaza guanosine, queuosine (Q), epoxyqueuosine (oQ), galactosyl queuosine (galQ), mannosyl queuosine (manQ), 7-cyano-7-deaza guanosine (preQ₀), 7-aminomethyl-7-deaza guanosine (preQ₁), archacosine (G⁺), 7-deaza-8-aza guanosine, 6-thio guanosine, 6-thio-7-deaza guanosine, 6-thio-7-deaza-8-aza guanosine, 7-methyl guanosine (m⁷G), 6-thio-7-methyl guanosine, 7-methyl inosine, 6-methoxy guanosine, 1-methyl guanosine (m¹G), N²-methyl-guanosine (m²G), N²,N²-dimethyl guanosinc (m²₂G), N^{2,7}-dimethyl guanosine (m^{2,7}G), N², N^{2,7}-dimethyl guanosine (m^{2,2,7}G), 8-oxo guanosine, 7-methyl-8-oxo guanosine, 1-methio guanosine, N²-methyl-6-thio guanosine, N²,N²-dimethyl-6-thio guanosine, alpha-thio guanosine, 2′-O-methyl guanosine (Gm), N²-methyl-2′-O-methyl guanosine (m²Gm), N²,N²-dimethyl-2′-O-methyl guanosine (m²₂Gm), 1-methyl-2′-O-methyl guanosine (m¹Gm), N^{2,7}-dimethyl-2′-O-methyl guanosine (m^{2,7}Gm), 2′-O-methyl inosine (1m), 1,2′-O-dimethyl inosine (m¹Im), 2′-O-ribosyl guanosine (phosphate) (Gr (p)), 1-thio guanosine, 06-methyl guanosine, 2′-F-ara guanosine, and 2′-F guanosine.

modRNA may include, for example, a non-natural or modified nucleotide. The non-natural or modified nucleotide may include, for example, a backbone modification, sugar modification, or base modification. The non-natural or modified nucleotide may include, for example, a base modification. In some embodiments, the base modification is selected from the group consisting of 2-amino-6-chloropurine riboside 5′ triphosphate, 2-aminoadenosine 5′ triphosphate, 2-thiocytidine 5′ triphosphate, 2-thiouridine 5′ triphosphate, 4-thiouridine 5′ triphosphate, 5-aminoallylcytidine 5′ triphosphate, 5-aminoallyluridine 5′ triphosphate, 5-bromocytidine 5′ triphosphate, 5-bromouridine 5′ triphosphate, 5-iodocytidine 5′ triphosphate, 5-iodouridine 5′ triphosphate, 5-methylcytidine 5′ triphosphate, 5-methyluridine 5′ triphosphate, 6-azacytidine 5′ triphosphate, 6-azauridine 5′ triphosphate, 6-chloropurine riboside 5′-triphosphate, 7-deazaadenosine 5′ triphosphate, 7-deazaguanosine 5′ triphosphate, 8-azaadenosine 5′ triphosphate, 8-azidoadenosine 5′ triphosphate, benzimidazole riboside 5′ triphosphate, N¹-methyladenosine 5′ triphosphate, N¹-methylguanosine 5′ triphosphate, N⁶-methyladenosine 5′ triphosphate, O⁶-methylguanosine 5′ triphosphate, N¹-methyl-pseudouridine 5′ triphosphate, puromycin 5′-triphosphate, and xanthosine 5′ triphosphate. Thus, according to some embodiments, the modRNA comprises N¹-methyl-pseudouridine 5′ triphosphate.

### Viral Vectors

Viral vector is an effective means of gene transfer. Examples of viral vectors suitable for use herein are retroviral vectors, lentiviral vectors, adenoviral vectors, adeno-associated viral vectors, and other vectors that can integrate into a chromosomal location within the host genome and provide stable expression of a gene of interest. Other vectors include episomal vectors, as well as engineered lentivirus vector variants that are non-integrative. A c-Maf encoding nucleic acid can be inserted into a viral vector and packaged in viral particles using methodologies known in the art. The recombinant viruses can then be isolated and incubated with a starting population of endothelial cells to deliver the c-Maf encoding nucleic acid to the endothelial cells.

### Lentiviral Vectors

Lentiviral vectors are vehicles for gene delivery that were originally derived from the human immunodeficiency virus type-1 (HIV-1) lentivirus. These vectors are defective for replication, and thus considered relatively safe, but are capable of stably integrating into the genomic DNA of a broad range of dividing and nondividing mammalian cell types. Engineered lentivirus vector variants that are non-integrative can also be used to deliver a c-Maf nucleic acid.

### Adeno-Associated Viral (AAV) Vectors

Adeno-associated viral (AAV) vectors are replication-defective, single-stranded DNA parvoviruses that require a helper Ad for their replication. Site-specific or random AAV vector integration into the host cell genome, in the absence of a helper virus, results in long-term transgene expression.

### Methods of Producing Induced Liver Sinusoidal Endothelial Cells (ILSECs)

In according with this disclosure, enhanced expression of the transcription factor MAF (c-Maf) in endothelial cells (particularly endothelial cells without a liver signature e.g., human generic endothelial cells such as HUVECs) promotes the generation of induced liver sinusoidal endothelial cells (ILSECs). Accordingly, this disclosure provides a method for producing induced iLSECs by enhancing the expression of c-Maf in endothelial cells.

In some embodiments, endothelial cells are modified to express c-Maf from an exogenously introduced nucleic acid to achieve enhanced expression of c-Maf.

In some embodiments, an exogenously introduced nucleic acid is a nucleic acid vector, such as a viral vector described herein, to express a c-Maf from the nucleic acid vector. In some embodiments, expression of c-Maf from the exogenously introduced nucleic acid is inducible. Inducible expression can be achieved by placing a c-Maf coding sequence under control of an inducible promoter, e.g., a metallothionine promoter, a glucocorticoid promoter, a progesterone promoter, and a tetracycline promoter. In some embodiments, inducible expression is achieved by using a Doxycycline expression system. The doxycycline (dox)-inducible Tet-On system can be used to control gene expression in mammalian cells. This system is based on the bacterial Tet operon, which has been modified and improved for its function in eukaryotic cells. The Tet-On system allows activation of gene expression by dox. In some embodiments, expression of c-Maf from the exogenously introduced nucleic acid is constitutive. Constitutive expression can be achieved by placing a c-Maf coding sequence under control of a constitutive promoter, e.g., an immediate early cytomegalovirus (CMV) promoter, a simian virus 40 (SV40) early promoter, a MoMuLV promoter, an Epstein-Barr virus immediate early promoter, a Rous sarcoma virus promoter, a human actin promoter, a human myosin promoter, and a human creatine kinase promoter. In some embodiments, expression of an exogenous c-Maf is transient, i.e., for a desired period of time, e.g., for about 5-9 days, or about 6-8 days, or about 6 days, about 7 days, or about 8 days. Transient expression can be achieved by, e.g., by removing the agent that is used to indue the expression (e.g., dox) or introducing an agent that inhibits or suppress the expression.

In some embodiments, an exogenously introduced nucleic acid is modified mRNA encoding a c-Maf.

In some embodiments, enhanced expression of c-Maf can also be achieved using a dead CrisprCas9-VP16 or any other dead CrisprCas9 System. For example, Cas proteins devoid of nucleolytic activity (dead Cas proteins; dCas) can be used to deliver an exogenous nucleic acid encoding a c-Maf to programmed sites in the genome.

An exogenous nucleic acid can be introduced into the recipient endothelial cells by methodologies known in the art. For examples, in embodiments where an exogenous nucleic acid is a viral vector, viral particles containing the viral vector carrying a c-Maf encoding nucleic acid can be prepared using suitable host cells and isolated viral particles can be incubated with recipient endothelial cells to achieve delivery of the viral vector into the endothelial cells. In embodiments where an exogenous nucleic acid is modified mRNA, the modified mRNA can be transduced into recipient endothelial cells by microinjection.

After an exogenous nucleic acid has been introduced into the recipient endothelial cells, the cells are cultured under conditions that allowed enhanced expression of c-Maf and facilitate development into iLSEC. In some embodiments, the cells are cultured in media containing Fetal Calf Serum and/or Human Serum. In some embodiments, the media also contain L-glutamax. In some embodiments, the media contain Heparin. In some embodiments, the media contain growth factors such as bFGF, EGF, Vegf, or IGF, or a combination thereof (e.g., a combination of bFGF, EGF, Vegf, and IGF). In specific embodiments, the media comprise 10% Fetal Calf Serum, 10% Human Serum, L-glutamax, Hepes, Antimicrobial antifungal, 100 ug/mlHeparin, 20 ng/ml bFGF, 10 ng/ml EGF, 10 ng/ml Vegf, and 10 ng/ml IGF.

In some embodiments, the enhanced expression of c-Maf is inducible and transient, e.g., for about 5-9 days (e.g., 5 days, 6 days, 7 days, 8 days, or 9 days). In some embodiments, the enhanced expression of c-Maf is inducible and transient for about 6-8 days (e.g., 6 days, 7 days, or 8 days).

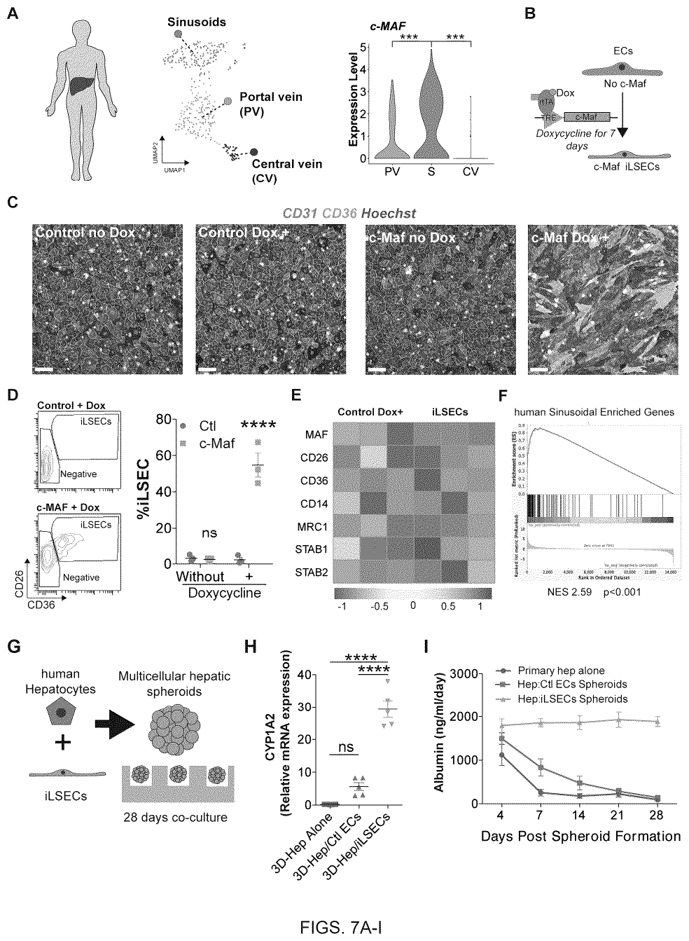

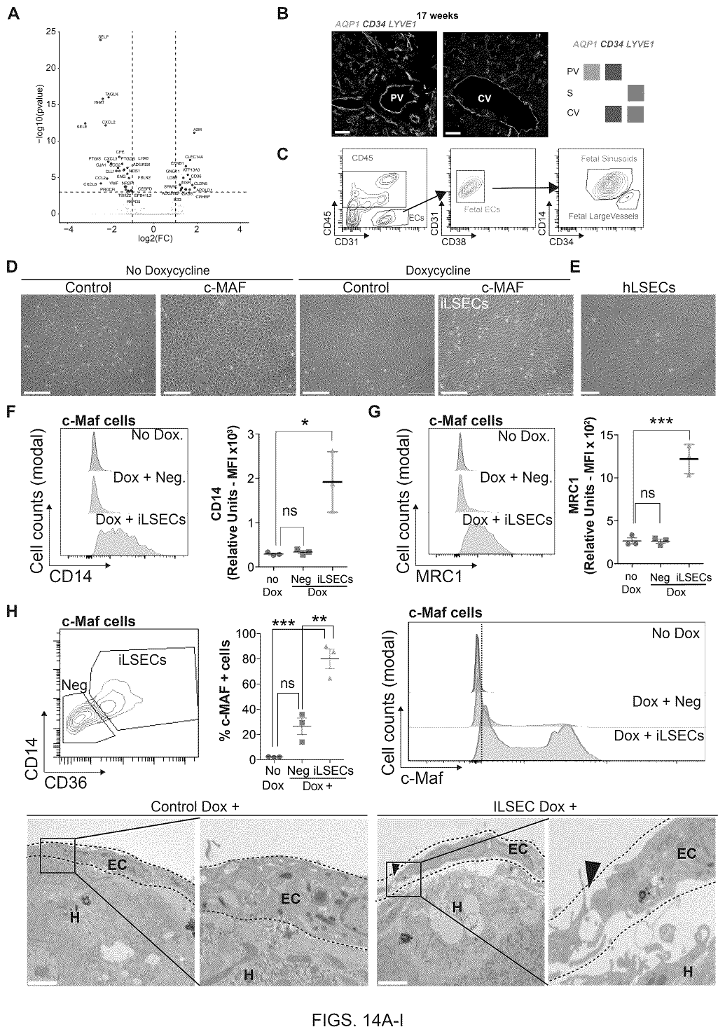

After enhanced expression of c-Maf for a period of time (e.g., for about 5-9 days such as 6-8 days) is achieved, the cells will change their morphology to the one of liver microvascular endothelial cells e.g., cobblestone morphology (scc, e.g., FIG. 7C) and display empty cavities that emulate fenestrations (see, e.g., FIG. 14I). Cells are then selected using CD26, CD36, and CD14 as cell surface markers, and cells that express at least two of the three markers on the cell surface, optionally also MRC1, are selected as representing iLSECs. The resulting isolated cell population is a substantially pure population enriched for cells displaying cell surface expression of the desirable markers (two or all three of CD26, CD36, and CD14).

In some embodiments, at least 85% cells in cell population display cell surface expression of two or three of CD26, CD36, and CD14. In some embodiments, at least 90% cells in cell population display cell surface expression of two or three of CD26, CD36, and CD14. In some embodiments, at least 95% cells in cell population display cell surface expression of two or three of CD26, CD36, and CD14. In some embodiments, at least 85% cells in cell population display cell surface expression of all three of CD26, CD36, and CD14, as well as MRC1. In some embodiments, at least 90% cells in cell population display cell surface expression of all three of CD26, CD36, and CD14, as well as MRC1. In some embodiments, at least 95% cells in cell population display cell surface expression of all three of CD26, CD36, and CD14, as well as MRC1. In some embodiments, at least 85% cells in cell population display cell surface expression of all three of CD26, CD36, and CD14, as well as MRC1 and VE-Cadherin.

iLSECs obtained herein as a result of enhanced expression of c-Maf and selection for at least two of the cell surface markers: CD26, CD30 and CD14 have great potential on the development of new approaches to test drugs on human adult hepatocytes or hepatocytes derived from embryonic stem cells or induced pluripotent stem cells.

Co-Culture of Hepatocytes in the Presence of an Isolated Population of iLSECs

It has been demonstrated herein that iLSECs generated in accordance with this disclosure are able to support long-term hepatocyte function, as demonstrated by CYP1A2 expression and maintenance of albumin production. Thus, enhanced expression of c-Maf is sufficient for the induction of liver sinusoidal identity and enables long-term sustainability of hepatocyte function. Therefore, in a further aspect, an isolated population of ILSECs is used to co-culture and maintain hepatocytes.

Primary adult human hepatocytes can be isolated from whole livers or resected liver tissues. A high flow-capacity perfusion device with a perfusion rate of up to 1-2 L/min is used for perfusion and isolation of hepatocytes from an adult human liver.

iLSECs and hepatocytes can be combined in any suitable tissue culture device to allow for interaction between the cells and formation of spheroids. ILSECs and hepatocytes are combined and co-cultured preferably in the presence of lethally irradiated fibroblast. In some embodiments, the spheroids formed can be maintained in medium that contains fetal bovine serum (FBS) (optional), Insulin-Transferrin-Selenium (ITS) at 1× concentration, and HGF at 50 ng/ml.

In some embodiments, co-culturing is carried out at an oxygen tension between 1% and 20%, e.g., at 20%.

In some embodiments, the co-culturing is carried out in a bioreactor, a tissue culture plate or a microfluidic device. In some embodiments, the co-culturing is performed in a decellularized matrix, a 3D scaffold, or a bioengineer matrix for the transplantation.

In some embodiments, the co-culturing is carried out for at least 20 days, at least 25 days, at least 26 days, at least 27 days, at least 28 days, at least 29 days, at least 30 days, or longer, e.g., up to 40 to 50 days.

iLSECs provided herein support a co-culture with hepatocytes for at least 20 days, at least 25 days, at least 26 days, at least 27 days, at least 28 days, at least 29 days, at least 30 days, or longer, e.g., up to 40 to 50 days. Hepatocytes cocultured with the iLSECs maintain their function, as assessed by quantifying the expression of CYP1A2—a key enzyme in liver metabolism—and the secretion of albumin, as exemplified herein below.

### Treatment of Liver Diseases

The liver is an immune-privileged organ that allows transplantation without matching HLA. Part of this capacity has been associated to the liver endothelial cells. It is believed that the iLSECs disclosed herein are useful for the adaptation of immune cells to treat graft vs host disease and other immunological problems.

These iLSECs may also be used in regenerative medicine to treat liver disorders by inoculating them into the body.

In addition, these iLSECs can also be used for the bioengineering of liver organs that contain the adequate liver vasculature and are able to properly support the hepatocytes.

These ILSECs can be used to treat the liver diseases e.g., acetaminophen toxicity, acute liver failure, alcoholic liver disease, liver cancer, cirrhosis, liver cyst, non-alcoholic fatty liver disease (NAFLD), and liver fibrosis.

In some embodiments, an isolated population of iLSECs is administered to a subject to treat a damaged liver or a liver disease in the subject.

In some embodiments, hepatocytes together with the isolated population of iLSECs are administered to a subject to treat a damaged liver or a liver disease in the subject.

In some embodiments, hepatocytes are cultured in the presence of an isolated population of iLSECs. Then the hepatocytes are isolated, and the isolated hepatocytes are administered to a subject to treat a damaged liver or a liver disease in the subject.

Administering of cells to the subject can be achieved by surgical or catheter implantation, subcutaneous injection, and infusion through an intravascular route. In some embodiments, the cells are administered to a site in the omentum or in the liver under the liver capsule through, e.g., implantation or subcutaneous injection.

### EXAMPLES

The present description is further illustrated by the following examples, which should not be construed as limiting in any way. The contents of all cited references (including literature references, issued patents, and published patent applications as cited throughout this application) are hereby expressly incorporated by reference.

### Example 1: Materials and Methods

### Animal Husbandry

All animal experiments were performed under the approval of Weill Cornell Medicine Animal Care and Use Committee. The c-Maf^{flox/flox} mice were obtained from Professor Carmen Birchmeier (Max Delbrück Center for Molecular Medicine). These mice have been crossed more than 10 times with C57/Bl6 mice and were crossed with the VEcadherin-Cre^{ER12} transgenic mice donated from Ralph Adams, also in C57/Bl6 background. For the developmental analysis, female c-Maf^{flox/flox} were crossed with male c-Maf^{flox/flox} VEcadherin-Cre^{ERT2} heterozygotes. The induction of the Cre was performed by the administration of a daily injection of 40 mg/kg of tamoxifen from E12.5 to E14.5. Tamoxifen was prepared in sunflower seed oil. Embryos were isolated at E16.5 and analyzed by flow cytometry, immunofluorescence, and RNA-seq.

The analysis of liver sinusoidal postnatal development was performed by induction of Cre with a solution of 2 mg/mL 4-hydroxytamoxifen from P2 to P4 and at P8. Mice were inoculated with 25 ul of this solution at P2 and 50 ul the remainder of the days. Littermate mice were analyzed at P15 when livers were processed for flow cytometry, sorting, or imaging as previously described.

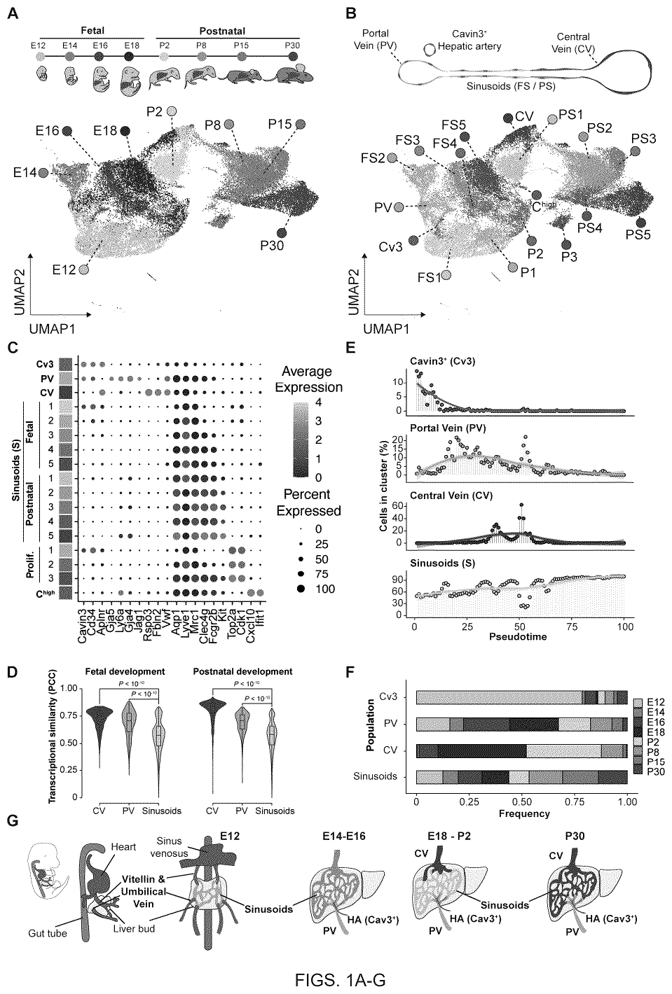

The analysis of liver sinusoidal markers in FIG. 1A was performed in C57/Bl6 mice obtained from Jackson Laboratories and analyze at 8 weeks of age. Adult mice were sacrifice in a CO₂ chamber and perfused with 15 mL of PBS and 10 mL of 4% paraformaldehyde (PFA). Livers were subsequently isolated and fixed overnight on 4% PFA. For the developmental analysis, females were sacrificed at the indicated timepoint, and the embryonic livers were isolated and fixed overnight with 4% PFA or processed for flow cytometry. The postnatal timepoint analysis was performed as indicated for the developmental timepoints, with the exception that mice were perfused with PBS and PFA from P8 onwards, similarly to the adults.

The analysis of adult mice was performed both for male and female mice, including c-Maf^{flox/flox} (controls) and c-Maf^{flox/flox}-VEcadherin-Cre^{ERT2+/-}, originating from the same litters. The induction of the deletion was performed by the administration of 6 doses of 40 mg/kg of tamoxifen over 9 days—3 days on, 3 days off, and 3 days on—after the mice were 4 weeks old. The mice were analyzed 4 weeks after the Tamoxifen administration, at 2 months of age.

Mice used for RNAseq analysis were intravitally label using a VEcadherin Alexa-647 antibody. Mice were euthanized using a CO₂ chamber and processed immediately thereafter. All mice were perfused with 10 mL of PBS. From the mice used for RNA-seq experiments, livers were extracted and stained for flow cytometry as described above. From the mice used for flow cytometry and staining, samples from the larger lobe of the liver were extracted and processed for flow cytometry, and after those mice were perfused with 10 mL of 4% PFA.

The development of liver fibrosis was induced by administrating CCl₄ twice per week at 25% dilution in sunflower oil. For the study of CCl₄ in control mice, C57/Bl6 mice were used from Jackson Laboratories at 8 weeks of age, inoculated with CCl₄ or only oil as a control. For the study of CCL₄ in c-Maf^{ΔEC} mice, the mice were treated with tamoxifen at week 4 as previously indicated and posteriorly with CCl₄ at week 8. The administration of CCl₄ was started at 2 months of age and performed for 4 weeks. Mice were euthanized 48 hours after the last injection was performed. Mice were perfused with 10 mL of PBS and 10 mL of 4% PFA solution. Livers were kept in 4% PFA solution overnight and transferred to 70% ethanol or 30% sucrose as described above.

### Cell Cultures

Human umbilical vein ECs (HUVECs) were extracted from umbilical cords obtained from New York-Presbyterian Hospital as previously described (Zhang et al. (2003), Blood 102, 2115-2121; Zhang et al (2004) J. Biol. Chem. 279, 11760-11766). Briefly, the umbilical cords were flashed with Hanks' Balanced Salt Solution (HBSS) and the inner side of the umbilical vein was incubated with 0.5% collagenase for 20 minutes as previously described. Cells were flashed out using HBSS and spun down in a centrifuge. Afterwards, cells were cultured on plates coated with 1% collagen using the following medium formulation: M199 basal medium (SH30253FS Fisher Scientific) supplemented with 10% fetal calf serum (Corning), 10% human serum (100-512 Gemini Bio-Products), 1× Corning glutagro Supplement (25-015-CI Corning), 1× Hepes (25-060-Cl Corning), 1× antimicrobial antifungal (2020 November Corning), 100 μg/mL heparin (H3393-Sigma), 20 mg/mL bFGF (100-18B Peprotech), 10 mg/mL EGF (AF-100-15 Peprotech), and 10 mg/mL Vegf (400-32 Peprotech). The medium was changed every 2 to 3 days.

HUVECs were transduced with control or c-Maf lentivirus at passage 2 and selected using 1 μg/mL of puromycin for 5 days, having a negative control on the plate. The induction of iLSECs was performed between passages 4 and 6 using the following protocol. Cells were grown until 50% confluence before administration of doxycycline on induction medium containing: M199 basal medium (SH30253FS Fisher Scientific) supplemented with 10% fetal calf serum (Corning), 10% human serum (100-512 Gemini Bio-Products), 1× Corning glutagro Supplement (25-015-CI Corning), 1× Hepes (25-060-Cl Corning), 1× Antimicrobial antifungal (2020 November Corning), 100 μg/mL heparin (H3393-Sigma), 20 mg/mL bFGF (100-18B Peprotech), 10 mg/mL EGF (AF-100-15 Peprotech), 10 mg/mL Vegf (400-32 Peprotech), 10 mg/mL IL6 (200-06 Peprotech), and 1 μg/mL doxycycline (04-0016 Stemolecule). The medium was changed every 2 days. Cells were induced over 7 days, starting on day 0, and analyzed by flow cytometry and immunofluorescence as explained above.

The study of the expression of c-Maf in mouse liver ECs in culture was performed using freshly isolated cells cultured for 48 hours in the presence of Bmp9, 116, or Lif. Mouse livers from healthy C57/Bl6 mice at 8 weeks of age were isolated, mineed in a solution of collagenase/dispase for 15 minutes, and cultured with anti-mouse CD31 magnetic beads for 1-2 hours. ECs were isolated using a magnet, washed five times with 1× MACS solution, and rinsed once in cold PBS. After that, cells were cultured in a medium containing: M199 basal medium (SH30253FS Fisher Scientific) supplemented with 2% fetal calf serum (Corning), 1× Corning glutagro Supplement (25-015-CI Corning), 1× Hepes (25-060-Cl Corning), 1× antimicrobial antifungal (2020 November Corning), 100 μg/mL heparin (H3393-Sigma), 20 ng/mL bFGF (100-18B Peprotech), and 10 ng/mL Vegf (400-32 Peprotech). For stimulation with 10 ng/ml of BMP9, 116 or Lif was used.

For the hepatocyte co-culture, c-Maf (CD31⁺ CD26⁺ CD36⁺) were sorted on an Aria II Flow sorter and co-cultured with the hepatocytes using a previously described co-culture system (Song et al., (2015) Sci. Rep.5, 16884). Briefly, human adult hepatocytes were obtained from Bioreclamation IVT. Hepatocytes and ECs were seeded on microwell plates together with lethally irradiated fibroblasts to promote their aggregation. The co-cultures were maintained for 28 days, with changes of medium every 2 to 3 days.

The production of lentivirus was performed on 293T cells grown on DMEM, 10% FBS, 1× L-glutamax, 1× Hepes, and 1× antimicrobial antifungal, similarly to previously described protocols (Lis et al., (2017) Nature 545, 439-445). Cells were transduced with the following plasmids: VSVG, REV, and RES from Cyagen, Inc., and the lentiviral plasmids pLenti empty control and pLenti c-Maf. Viral titers were calculated using a p24 assay (632200-Clontech).

### Flow Cytometry and Cell Sorting

Isolation of cells to be used in flow cytometry and cell sorting analysis were performed as follows. Liver samples were isolated from the mice, mineed, and incubated with collagenase A (25 mg/ml) and dispase II (25 mg/mL) at 37° C. for 10 to 15 minutes. Cells were filtered using a 100 μm filter and spun down at 300 g for 5 minutes. Samples were RBC lysed for 5 minutes on ice using RBC lysis solution (Biolegend). After 5 minutes, samples were rinsed with PBS and spun down. Liver samples were stained using 1× MACS buffer solution. Cells were first incubated with the FC-quenching antibody before staining.

From the human livers, the non-parenchymal cell (NPC) fraction was obtained from the laboratory of Dr. Robert Schwartz. Samples were processed similarly to mouse liver samples after the RBC lysis step. The human fetal liver sample was processed similarly to the mouse livers from the beginning of the protocol.

10× Chromium scRNA-Seq Analysis

Developmental trajectory experiment: Mouse endothelial cells (ECs) were sorted as indicated above under the Flow cytometry and Sorter description. The sorted CD45^{neg} CD31⁺ cells were transfer to the Genomics Core from Weill Cornell Medicine to proceed with the Chromium Single Cell 3′ Reagent Kit v2 (10× Genomics, product code #120267) cell protocol using 10× Genomics' Chromium Controller. A total of 8,000 to 10,000 cells were loaded into each channel of the Single-Cell A Chip from each sample. Samples were sequenced at the recommended depth of 50,000 reads per cell in an Illumina HiSeq 4000 sequencer. Sequencing output was de-multiplexed and post-processed following the 10× genomics custom pipelines using the cellRanger (v4.0.0.) software. Raw base calls were de-multiplexed with the mkfastq command, followed by alignment to the mm10 reference genome. Barcode and unique molecular identifier (UMI) counting was performed using the cellranger command with default parameters. Cell barcodes with UMI>500 and mitochondrial reads <20% were retained for downstream analysis. After filtering, 5,081+/−1,014 (mean±/−standard deviation) cells per sample were obtained, with a total of 40,655 cells recovered.

Samples were analyzed using the software Seurat (v 3.2.3) (Butler et al., (2018) Nat. Biotechnol. 36, 411-420; Stuart et al., (2019) Cell 177, 1888-1902.c21). Normalization was performed using the SCTransform function, regressing out the following variables: total number of UMIs per cell, percentage of mitochondrial UMIs, S phase score and G2M score. Following normalization, principal component (PC) analysis was performed. Next, the amount of variance explained by the top 30 principal components was verified. The initial 24 PCs showed above 0.5% of variance explained. Visual inspection of PCs above 25 showed a more homogeneous expression of the composing genes with no clear separation and a decrease of the variance explained. Therefore, PCs up to PC24, were retained for downstream analysis. Clusters were defined using the FindNeighbors function in the PC space, followed by the FindClusters function with a resolution of 0.8, as it resulted in biological relevant clusters with clear marker genes that were manually curated for cell type annotation. Cluster marker genes were identified using the FindAllMarkers function with the following parameters: log.fc.threshold=0.25, min.pct=0.1 and only.pos=TRUE.

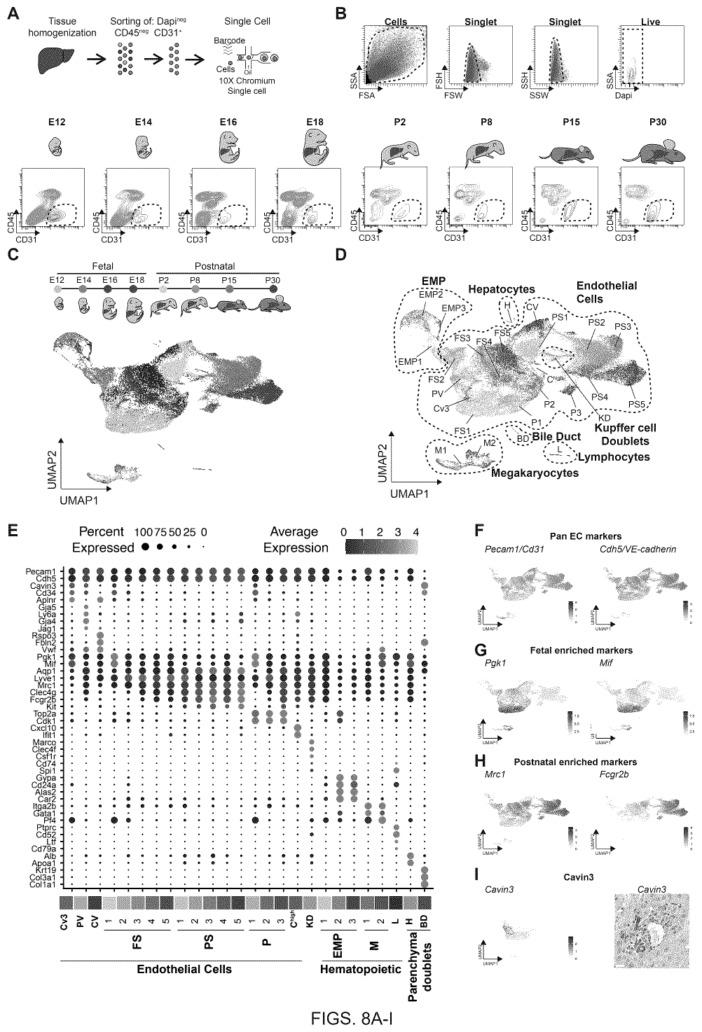

For RNA velocity analysis, loom files were generated using velocyto (v0.17.17) (La Manno et al., (2018) Nature 560, 494-498). using the run 10× function. RNA velocity calculation was performed using sc Velo (v0.2.2) (Bergen et al., (2020) Nat. Biotechnol 38, 1408-1414). Genes were filtered and normalized using the filter_and_normalize function with the following parameters: min_shared_counts=100 and n_top_genes=2000. First and second order moments (means and uncentered variances) were computed using the moments function, with n_pcs=30 and n_neighbors=30. Gene dynamics were recovered using the recover_dynamics function and RNA velocities were calculated with the velocity function setting mode=‘dynamical’. For downstream analysis of vascular endothelial cells, endothelial cell clusters were selected based on the expression of the markers Cdh5, Pecam1 and the absence of Ptprc as indicated in: FIG. 1 and FIG. 8 for the developmental trajectory. RNA velocity vector field was estimated using the velocity_embedding_stream with basis=‘umap’ and min_mass=0. Estimation of RNA velocity-based pseudotime was performed using the velocity_pseudotime function with default parameters. For estimation of transition probabilities across developmental time points, PAGA graph abstraction (Wolf et al., (2019) Genome Biol. 20, 59) was performed using the paga function, setting the developmental time points as the ‘groups’ parameter.

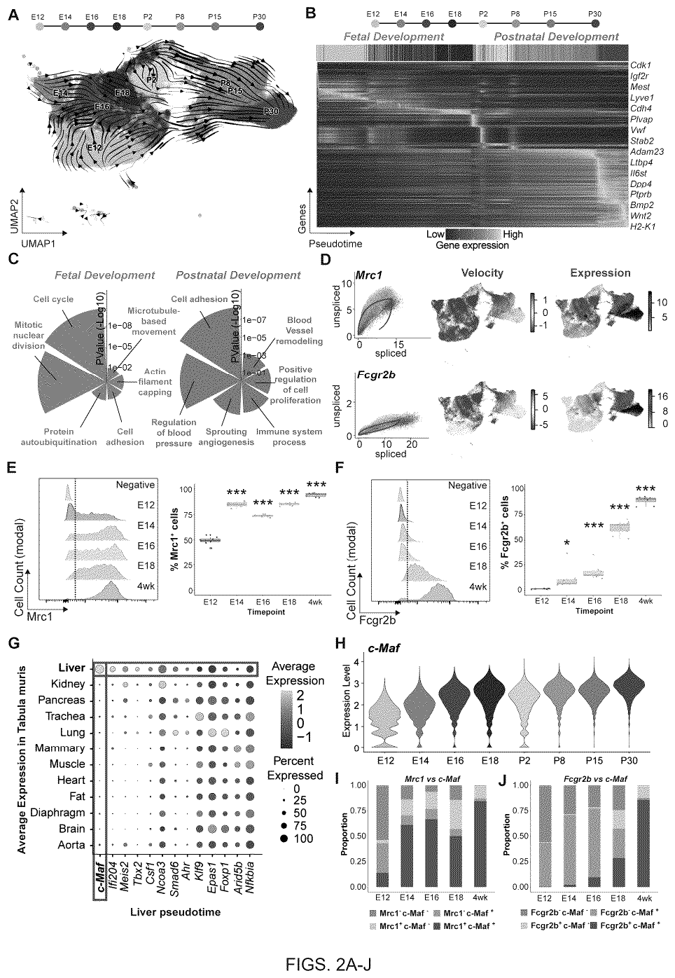

The identification of the transcription factors from the pseudotime analysis in FIG. 2 was performed based on the TTRUST version2 mouse transcription factor list (Han et al., 2018). Comparison of all liver endothelial transcription factors was performed by downloading the Tabula muris database and calculation of the liver enriched genes compared to endothelial cells from other organs. The liver enriched list of genes was compared to the transcription factors identified from the pseudotime analysis using TTRUST version2.

P15 control and c-Maf^{ΔEC} experiment: For the analysis of P15 samples from Control and c-Maf^{ΔEC} mice was performed over a mixed sample containing male and female cells coming from each sample. Count matrices were generated using the cellRanger software (v4.0.0.) as described above, and Seurat (v3.2.3) was used for downstream analysis. Samples were processed and split based on the expression of the following sexual dimorphism genes: male: Ddx3y, Eif2s³y, Gm29650, Kdm5d and Uty; and female: Xist. These genes were excluded from further analysis to avoid enrichment based on sexual dimorphism. Data was split based on the sex of the mice and normalized using the SCTransform function. In order to perform integration, 3,000 integration features were selected using the SelectIntegrationFeatures function and excluding the sexual dimorphism genes. Next, the PrepSCTintegration function setting assay=′SCT′ was applied, and integration anchors were defined using the FindIntegrationAnchors with dims=1:40. Final integration was performed using the IntegrateData function with normalization.method=‘SCT’. Following integration, PCA analysis was performed using the RunPCA function, and 20 PCs were retained for downstream analysis. Cell clustering was performed with the FindNeighbors function followed by the FindClusters function with resolution=0.5. Uniform manifold approximation (UMAP) dimensionality reduction for visualization was performed using the RunUMAP function.

For RNA velocity analysis, loom files were generated using (v0.17.17) (La Manno et al., (2018) Nature 560, 494-498) as described above, and sc Velo (v0.2.2) (Bergen et al., (2020) Nat. Biotechnol. 38, 1408-1414) was used for downstream analysis. Endothelial cells were retained based on expression of the markers Cdh5, Pecam1 and the absence of Ptprc and downstream RNA velocity analysis was performed as described above.

ddSeq scRNA-Seq Analysis

Human liver ECs were sorted as indicated above and transferred to the Genomics Core Facility at Weill Cornell Medicine to proceed with Illumina Bio-Rad SureCell WTA 3′ Library Prep kit using the Bio-Rad ddSEQ Single-Cell Isolator system. Briefly, according to manufacturer's instructions (Illumina, cat #20014280), the sorted cells were washed with 1× PBS+0.1% BSA, counted by Bio-Rad TC20 Cell Counter, and cell viability was assessed. A total of 12,000 cells and barcode mixes were loaded into each channel of the cartridge to generate the droplets on the ddSeq Single Cell Isolator, from which a total of 769 cells were recovered. After the first strand was synthesized in droplets, individual droplets were disrupted. The second strand cDNA synthesis was carried out and the RNA template was removed. Using the Illumina Nextera SureCell transposome kit, cDNA was fragmented simultaneously and tagged with adapter sequences in a single step. Following PCR amplification, cDNA libraries were assessed using the Agilent Technology 2100 Bioanalyzer and sequenced on the Illumina NextSeq 500 sequencer using the high output mode with 150 cycle kit. FASTQ files were then generated in the Illumina BaseSpace SureCell Single-Cell System.

Cell barcodes with UMI<500 and UMI>15,000 were filtered out. Mitochondrial genes were removed as a potential confounder of clustering. Data was analyzed using the Seurat R package (v 3.2.3) (Butler et al., (2018) Nat. Biotechnol. 36, 411-420; Stuart et al., (2019) Cell 177, 1888-1902.e21). Raw UMI counts were log-normalized using the NormalizaData function with the following parameters: normalization.method=“LogNormalize” and scale.factor=1,000. Variable genes were selected by using the FindVariableGenes function with the following parameters: mean.function=ExpMean, dispersion.function=Log VMR, x.low.cutoff=0.3; x.high.cutoff=3; y.cutoff=0.5. Data was then scaled and UMI number was regressed out as potential confounders of clustering using the Sca1eData function. Following normalization, PC analysis was performed followed by JackStraw analysis to determine the significant PCs to be used in downstream analysis. A total of 11 significant PCs were retained for downstream analysis. Clusters were defined using the FindNeighbors function in the PC space, followed by the FindClusters function with a resolution of 0.4.

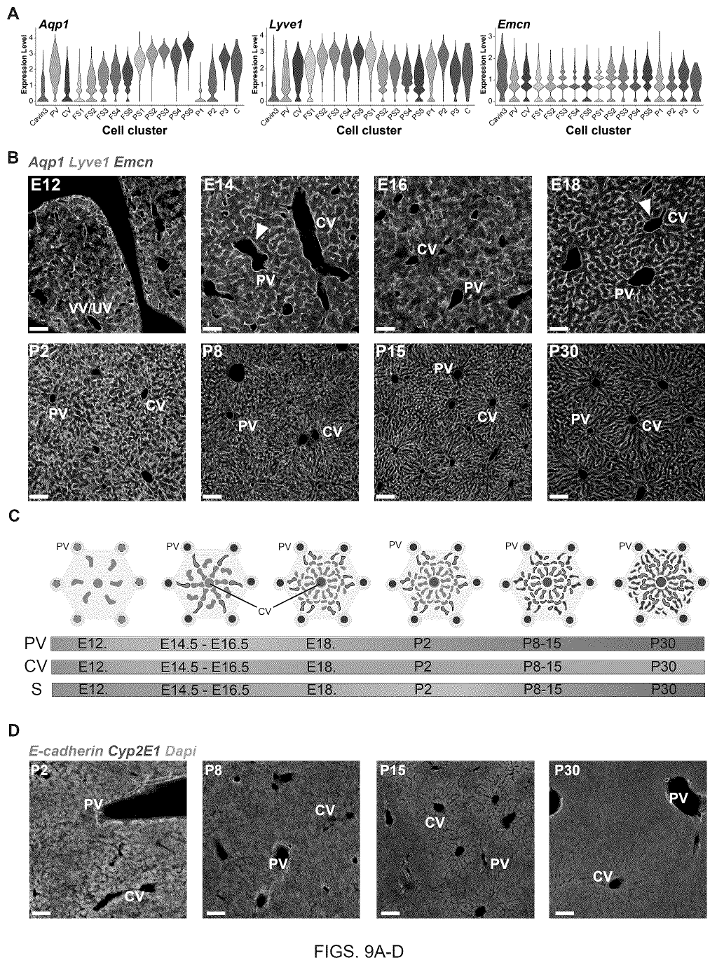

A total of four clusters were identified. The genes enriched in the four different clusters of ECs were identified using the FindAllMarkers function with the following parameters: only.pos=TRUE; min.pct=0.25; thresh.use=0.25. One of the clusters corresponded to contamination of CD45⁻ CD31⁺ CD38⁺SCD1⁺ plasma cells, which were removed for analysis of ECs in FIGS. 1-7 but indicated in FIGS. 8-9. UMAP plots were generated using the RunUMAP function with the significant PCs.

### Bulk RNA Sequencing

Mouse ECs from control and Maf^{ΔEC} were sorted as indicated above using the markers CD45⁻ CD31⁺ VEcadherin⁺. Human cells overexpressing c-Maf were isolated by sorting the fraction of cells positive for CD31, CD26, and CD36. Control cells were sorted based on the expression of CD31, since these cells do not express either CD26 or CD36. RNA was isolated using the RNA isolation Mini Kit from Qiagen (74104-Qiagen), following the instructions from the manufacturer. At least 100 ng of total RNA from freshly harvested cells was isolated and purified using Qiagen's RNeasy Mini Kit. RNA quality was verified using an Agilent Technologies 2100 Bioanalyzer prior to sequencing. RNA library preps were generated and multiplexed using Illumina's TruSeq RNA Library Preparation Kit v2 (non-stranded and poly-A selection). 10 nM of cDNA was used as input for high-throughput sequencing via Illumina's HiSeq 4000, producing 51 bp paired-end reads.

### NGS Data Processing and Statistical Analysis

Sequencing reads were de-multiplexed (bcl2fastq v2.17), checked for quality (FastQC v0.11.5), and trimmed/filtered when appropriate (Trimmomatic v0.36). The resultant high quality reads were mapped (TopHat2 v2.1.0; Bowtie2 v2.2.6) to the transcriptome sequence reference of the UCSC mm10 genome build. Gene counts were quantified using the Python package HTScq (v0.11.1). Transcript abundance measures (FPKM values) were quantified using Cufflinks (v2.2.1). Gene-level differential expression analysis was performed using the Bioconductor R package DESeq2 (v1.22.2).

### Transcriptome Data Analysis

Transcriptomic data analysis was summarized in the forms of heatmaps and gene set enrichment plots. Heatmaps were generated using the CRAN R package pheatmap (v1.0.12). GSEA plots were generated using the R scripts available from the Broad Institute (GSEA v1.0).

### Processing of ATACseq Files

Liver endothelial cell ATACseq were downloaded from GSE154828 (Winkler et al., (2021) J. Hepatol. 74, 380-393). Fastq files were checked for quality using FastQC and processed using adapters trimmed with Cutadapt wrapper Trim Galore. Reads were aligned with bowtie2, and duplicated reads were marked with Picard. Reads with multimap, duplicates and low-quality reads were removed. Remaining reads were shifted on positive/negative strand and peaks were identified using MACS2. Motif analysis was performed using HOMER and scan against c-Maf motif locations. c-Maf peaks were extracted as peaks that overlap a c-Maf motif region after removing those in blacklisted regions. Genes flanking the peaks were determined using a 10 kb window from the peak with Bedtools. From this list of genes, it was looked at which were proximal to up and down regulated differentially expressed genes.

### Immunofluorescence Protocol

Mouse tissues embedded in OCT compound were section on a cryostat at 20 μm thickness and kept at −80° C. Sections were thawed at room temperature and washed three times with a PBS solution for 5 minutes. Tissues were permeabilized using 0.1% Triton solution for 10 minutes and washed with PBS 3 times for 5 minutes. Afterwards samples were incubated for 30 minutes with blocking solution (1× PBS, 5% donkey Scrum, 0.1% Triton). Tissues were incubated for two days at 4° C. with the primary antibodies at a dilution of 1:100 in blocking solution. Primary antibodies were wash three times with PBS for 5 minutes. Secondary antibody staining was performed for three hours at room temperature in blocking solution. Secondary antibodies were washed three times in PBS for 10 minutes. Samples were mounted on Fluoroshield with Dapi (F6057, Sigma) and imaged using a Zeiss 710 confocal microscope.

Human adult livers were obtained from the histopathology department at Weill Cornell Medicine. All samples were deparaffinized following this procedure: 2×3 minutes methanol, 1 minute 50% methanol 50% ethanol, 1 minute 95% ethanol, 1 minute 80% ethanol, 1 minute 75% ethanol, 1 minute 50% ethanol, 1 minute 25% ethanol, 3 minutes PBS. After that, samples were incubated with 1× Buffer B from Electron Microscopy Science (62706-11) and posteriorly boiled on a Retriever Thermal Slide Processor (Electron Microscopy Science) for 1 hour. After cooling down samples were washed three times for 5 minutes with PBS and incubated with 0.1% PBS-Triton for 10 minutes. Samples were washed three times for 5 minutes with PBS and incubated with blocking solution for 30 minutes. Primary antibodies were incubated with blocking solution at a concentration of 1:100 overnight. After washing three times for 5 minutes with PBS, secondary antibody staining was performed at 1:500 dilution with blocking solution for 3 hours at room temperature. Samples were mounted on Fluoroshield with Dapi (F6057-Sigma) and imaged using a Zeiss 710 Confocal Microscope.

Human fetal liver was obtained from Advanced Bioscience Resources, Inc. Small pieces of this sample were incubated on 4% PFA overnight, washed with PBS twice for 5 minutes, and kept in a 30% sucrose solution for 3 days before being included in OCT. The staining for this sample proceeded similarly to the ones from the mouse tissues.

### Histology

All organs were fixed overnight in 4% PFA at 4° C. and cleaned three times with 1×PBS for 10 minutes each time. After this step, the organs used for paraffin histology were kept in 70% ethanol and were sent to Histoserv, Inc. to be processed for: hematoxyline, cosin, and Masson's trichrome staining. The livers used for immunofluorescence were maintained for 3 days in a 30% Sucrose solution at 4° C. After this time organs were included in OCT. Histopathology analysis was conducted to evaluate the degree of liver damage and a pathologist blindly analyzed necrosis. Histological images were acquired on a Zeiss Axio Observer Z. 1 microscope with a 20×/0.8NA objective and a AxioCam 305 color camera. Quantification of fibrosis deposition was performed using the Zeiss Zen 2.6 image analysis package, using tiled images of whole liver sections. The amount of liver fibrosis was divided by the surface are of the liver quantified for all images.

### Lacunarity and Fractal Analysis

The branching and space-filling of the liver structure display a hierarchical self-similarity quantifiable with fractal mathematics, in particular the parameters of fractal dimension (D) and lacunarity (A) (Adelson et al, (2021) Microvasc. Res. 138, 104205). D is a ratio indicating the change in complexity of the structural pattern with scale, while A quantifies the size and spatial distribution of gaps between branches in the pattern. Liver images were analyzed in ImageJ in order to quantify these fractal properties. Color-binarized two-dimensional confocal microscopy images were loaded into ImageJ using the FracLac plug-in.

D of each liver image was quantified using a box-counting method, with a grid of known box dimension (r) overlaid on each image. The number of boxes containing structural pixels (N) was counted. This procedure was repeated for all images for each of 100 grids of varying r (5 to 1,742 pixels wide), with N decreasing exponentially as box size increased.

Plotting r versus N reveals D, which approximately equals to the negation of the slope of the best-fit regression line. The relevant equation is:

$$D = - log ( N ) / log ( r )$$

Λ was measured using a similar method, a sliding box scan algorithm, which involves sliding boxes over the image in an overlapping pattern. The mean (μ) and standard deviation (σ) of pixels per box was calculated for each r, and Λ was approximated using the following equation (an average Λ was then calculated based on all r used):

$$Λ = {( σ / μ )}^{2}$$

### Matrix Cellulose Assay

Analysis of the hemato-progenitor cells contained in the livers from control and c-Maf endothelial deficient mice were performed at postnatal day P15. Livers were processes as previously indicated for flow assisted cytometry sorting analysis. Hematopoietic cells were identified as a CD45⁺ CD31⁻ population, from which 90.000 cells were sorted. Hematopoietic cells were transfer to 3 mL matrix cellulose assay tubes with MethoCult GF m3434 (#03444 StemCell Technologies), from which 2 mL were plated and cultured for 14 days. After 2 weeks, the number of colonies was counted for all plates and the average of the two plates per mouse were calculated, providing the total colony forming units per 30,000 cells.

### Cloning

c-Maf was PCR amplified from the plasmid (VB 170606-1099bva) from Vectorbuilder, Inc (Cyagen Bioscience) and cloned into a pZip lentiviral plasmid carrying a Puromycin resistance and RtTa. The PCR product and Lentiviral plasmid were digested with the enzymes BamHI and AsisI and Ligated. Individual clones were validated by sequencing.

Quantitative Real Time Reverse Transcription Polymerase Chain Reaction (qRT-PCR)

Spheroid aggregates of iLSECs and hepatocytes were washed with PBS three times and then collected into Eppendorf tubes. After centrifugation at 1000 rpm for 5 minutes, the pellets were lysed with RLT buffer and total RNA was isolated using an RNAcase Kit (Qiagen). Isolated RNA was then treated with DNase (NEB) and RNA cleaned up with the RNA Clean and Concentrator kit (Zymo Research). 2 μg of RNA was used for cDNA synthesis (iSCRIPT cDNA synthesis kit, Biorad). qRT-PCR was performed using SYBR Green PCR (Biorad) on a Biorad CFX PCR machine. The gene expression data was normalized using β-actin as a housekeeping gene.

### Albumin Secretion

Albumin secretion was measured in culture medium. The medium was collected and replaced with fresh medium every 2 days. The collected medium was centrifuged at 1000 rpm for 5 minutes and the supernatant was stored at −20° C. Secreted albumin was quantified by an enzyme-linked immunosorbent assay (ELISA) kit using sheep anti-human albumin antibodies (Bethy Labs) and horseradish peroxidase detection (3,3′,5,5′-tetramethylbenzidine, Invitrogen).

### Quantification and Statistical Analysis

Statistics were calculated using R software, the R package “ggpubr” and Graph Pad. Groups of 2 different conditions were compared using T-test. Statistical significance is shown as: *p<0.05, **p<0.01, ***p<0.001. Boxplots indicate the medium, data within the boxplot shows the interquartile range, whisker shows the minimum and maximum values used for the boxplot, values outside the whiskers were consider as outlawyers by R. Barplots represent medium of the data±SEM. All information related to the analysis of each panel can be found in the figure legend. Quantification of liver fibrosis was performed using Zeiss Zen 2.6 image analysis package. Total liver fibrosis was divided by the total surface are of the liver for all images.

### Example 2: Diversification of Liver Vasculature is Developmentally Specified

Upon differentiation, liver endothelial progenitors acquire their specialization by expressing defined surface receptors and cytokines to meet the demands of the co-localized liver parenchyma. While fetal and adult liver sinusoids are phenotypically distinct (Bankston and Pino, (1980) Am. J. Anat. 159, 1-15; Barberá-Guillem et al., (1986) J. Ultrastruct. Mol. Struct. Res. 97, 197-206; Nonaka et al., (2007), Dev. Dyn. 236, 2258-2267) it is unclear how this heterogeneity is progressively established and maintained across fetal and postnatal development. To address this, scRNA-seq were performed on sorted ECs defined as CD45^{neg}CD31⁺ cells every two days from embryonic day 12 to 18 (E12-E18) and at postnatal day 2, 8, 15 and 30 (P2, P8, P15 and P30, respectively) of development (FIG. 1A, FIG. 8A-B). The analysis identified a total of 17 EC clusters (FIG. 1B), with contamination of hematopoietic cells and a small fraction of parenchymal cells (FIG. 8C-E).

The EC populations were identified as CD31⁺ CDH5⁺ CD45^{neg} (FIG. 8E, F), and cell cluster identity was defined based on marker gene expression: Cavin3⁺ (Cav3⁺) by expression of Cavin3, Portal Vein (PV) by expression of Gja5, Cd34, Ly6a, Gja4, Jag1 and Vwf, Central Vein (CV) by expression of Rspo3, Fbln2, Vwf, sinusoids(S) expressing Cd34, Mrc1, Fcgr2b, Clec4g and Kit, a proliferative cell cluster expressing Top2a and Cdk1, and a Cxcl10 high (C^{high}) cluster with expression of Cxcl10 and Ifit1 (FIG. 1C and FIG. 8E-I). Within the sinusoidal group, a unique expression pattern of undifferentiated markers was observed such as: Cd34, Pgk1 and Mif compared to adult differentiated markers, such as Aqp1, Mrc1, Fcgr2b, Clec4g and Kit (FIG. 1C and FIG. 8E, G, H). Notably, the sinusoidal EC population displayed the lowest degree of internal transcriptional similarity within the developmental stages (FIG. 1D), rendering sinusoids the most transcriptionally diverse cell type across the landscape of vascular capillary development compared with large vessel clusters Portal Vein and Central Vein.

Angiogenic signals arising from the liver bud at E10-E12 induce its vascularization from the progenitors within the Vitellin Vein, the Umbilical Vein and Sinus Venosus (DeSesso, (2017) Reprod. Toxicol. 70, 3-20), facilitating contribution from the endocardium to liver endothelium (Zhang et al., (2016) Nat. Genet. 48, 537-543). A decrease in the frequency of the Cavin3⁺ cluster was noted at pseudotimes corresponding to later developmental stages (FIG. 1E-F), although a small percentage was present at postnatal stages and these cells were identified as Hepatic artery cells based on Cavin3 staining in the Protein Atlas (FIG. 81). The Portal Vein cluster increased in frequency shortly after the decrease of the Cavin3⁺ cluster (FIG. 1E-F). Notably, the establishment of the Central Vein cluster occurred during late fetal and early postnatal development (E18-P2; FIG. 1E-F). While the Central Vein and Portal Vein signatures occurred during restricted developmental windows, sinusoid frequency steadily increased over time (FIG. 1E-F).

Based on protein and RNA expression patterns, the developmental changes of the Portal Vein and adult sinusoidal marker Aqp1, the fetal enriched marker Lyve1 and the Central Vein marker Emen were characterized (FIG. 9A-C). scRNA-seq analyses revealed specific vascular populations during restricted developmental times with Aqp1 induction by the Portal Vein at E14 and Endomucin (Emcn) by the Central Vein at E18 (FIG. 9A-C). Moreover, establishment of Central Vein induced the hepatocyte co-zonation as measured by CYP2E1 at stage P2 (FIG. 9D). This is in agreement with the requirement of Rspo3 angiocrine secretion from Central Vein regulating early liver postnatal function (Boj et al., (2012) Cell 151, 1595-1607; Planas-Paz et al., (2016) Nat. Cell Biol. 18, 467-479; Rocha et al., (2015) Cell Rep. 13, 1757-1764). These data suggest a temporal specification of each liver vascular component and defines sinusoids as the most transcriptionally diverse EC type during liver development (FIG. 1G).

### Example 3: Temporal Dynamics of Liver Sinusoidal Specialization

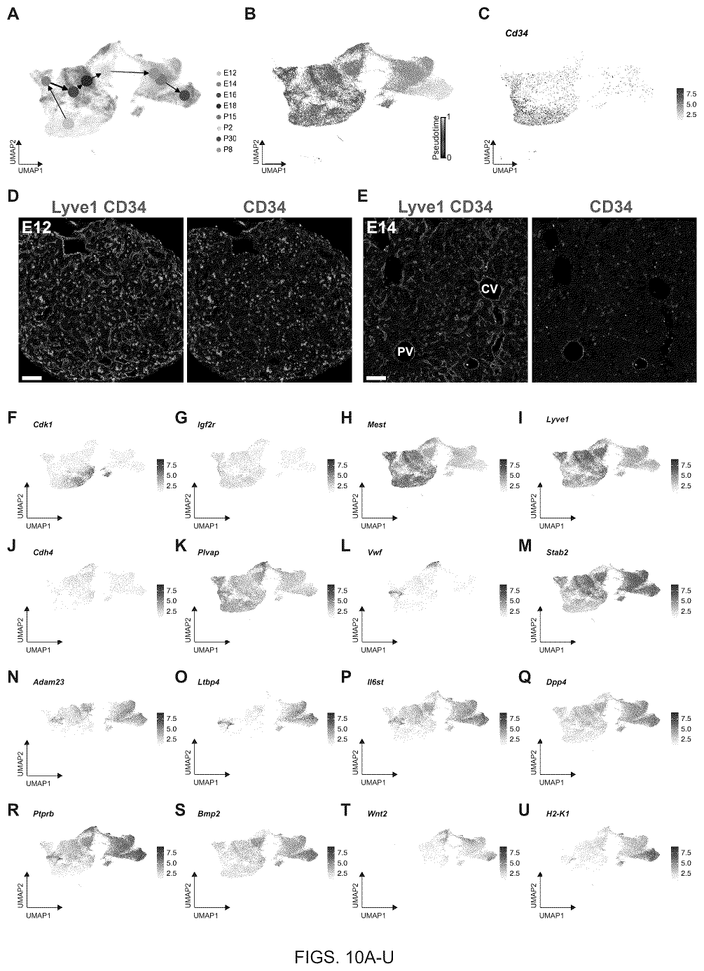

To uncover transcriptional dynamics during liver vascular differentiation, RNA velocity analysis (Bergen et al., (2020) Nat. Biotechnol. 38, 1408-1414) on EC fractions was performed (FIG. 2A). Estimation of transition confidence between developmental stages using graph abstraction (Wolf et al., (2019) Genome Biol. 20, 59) recapitulated the expected in vivo development (FIG. 10A). Ordering of the cells along RNA velocity-inferred pseudo time revealed two main groups of driver genes (fit likelihood >0.17), associated with either fetal or postnatal development (FIG. 2B and FIG. 10B). At early timepoints (E12), undifferentiated EC progenitors expressed Lyve1 and Cd34 as observed by scRNA-seq and immunofluorescence analysis, although Cd34 became restricted to the Portal Vein (PV) at E14 (FIG. 10C-E). Sinusoids were shown to acquire the capacity to express angiocrine factors Igf2r, Dpp4, Bmp2, Wnt2 and Ptprb during development (FIG. 2B and FIG. 10F-T). The genes driving the fetal developmental process were enriched for markers of cell proliferation, active protein turnover, and cell migration, among others (FIG. 2C). Postnatal development driver genes were enriched for vascular maturation markers, including regulation of blood pressure, immune system, blood vessel remodeling, and secretion of cytokines involved in angiogenesis and cell proliferation (FIG. 2C). Active transcription of adult sinusoidal markers, such as Mrc1 and Fcgr2b measured by RNA velocity was observed at early time points during fetal development and increased during postnatal stages (FIG. 2D). Thus, an initial colonization of the liver bud by CD34⁺ endothelial progenitors lead to a progressive differentiation into the liver vasculature.

To verify the progressive acquisition of sinusoidal identity, fluorescence cytometry analysis of the sinusoidal markers Mrc1 and Fegr2b during fetal development was performed and compared it at 4 weeks postnatal stage. Mrc1 was detected early at E12 (FIG. 2E) and precedes the Fcgr2b appearance at E18 (FIG. 2F). Therefore, liver sinusoidal EC fate is specified in a dynamic temporal sequence initiated during development and completed postnatally.

### Example 4: Identification of c-Maf as a Liver Sinusoidal Specific Transcription Factor

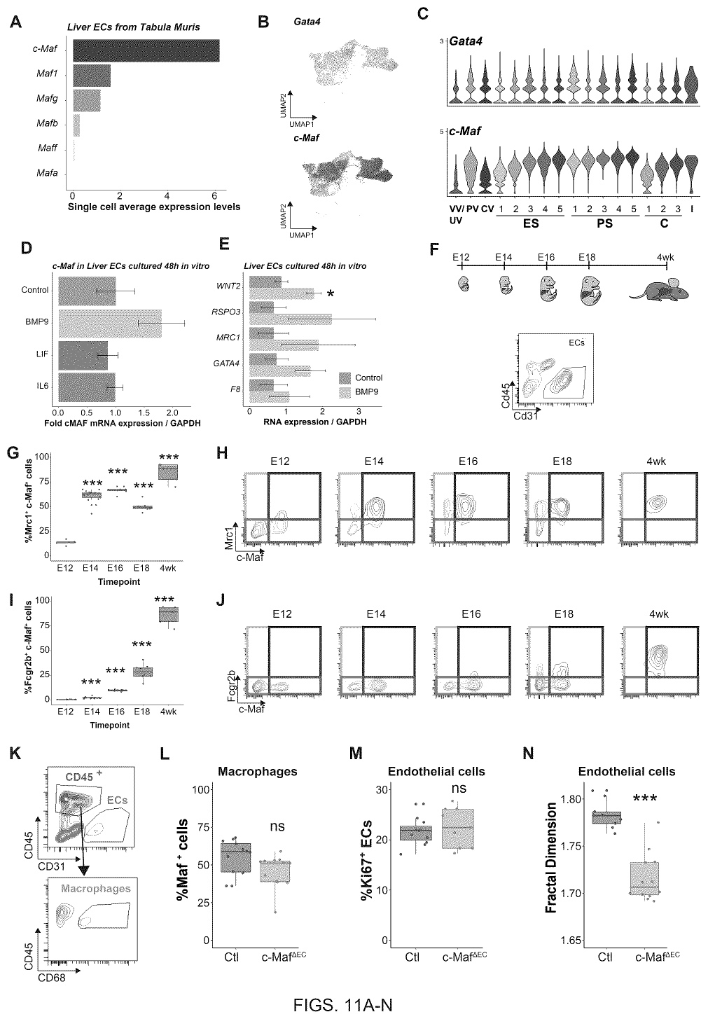

To elucidate the molecular determinants of sinusoids, transcription factors identified as drivers (fit likelihood >0.17) were analyzed in the RNA velocity analysis. To define those transcription factors specific to liver endothelium, differential gene expression analysis across tissues from the Tabula Muris database (Tabula Muris Consortium et al., (2018) Nature 562, 367-372) were performed. c-Maf was found to be the top-enriched (FDR<10⁻¹⁰; fold change=3.6) transcription factor in liver ECs (FIG. 2G), with a continuous increase over time (FIG. 2H), being the most highly expressed member of the Maf family within liver sinusoids (FIG. 11A). Notably, while Gata4 also mediates liver sinusoidal development (Géraud et al., (2017) J. Clin. Invest. 127, 1099-1114); Gata4 widespread homogenous expression across liver EC types and developmental stages suggests a wider functionality as compared to c-Maf (FIG. 11B, C).

Expression of Bmp9 (Desroches-Castan et al., (2019a) Hepatology 70, 1392-1408), LIF and IL6 (Giordano et al., (2015) EMBO J. 34, 2042-2058); Yang et al., (2005) J. Immunol. 174, 2720-2729). have been associated with c-Maf expression. Treatment of liver EC cultures with Bmp9 in vitro, but not with LIF or IL6 increased c-Maf expression (FIG. 11D), along with other liver endothelial genes (FIG. 11E). To uncover the contribution of c-Maf to liver vascular development, flow cytometry analysis of c-Maf and the sinusoidal markers Mrc1 and Fcgr2b from fetal E12.5 to E18.5 and in 4 weeks postnatal stage were performed (FIG. 11F). An increase in co-expression of c-Maf, Mrc1 (FIG. 2I and FIG. 11G, H) and c-Maf with Fcgr2b (FIG. 2J and FIG. 11I, J) through the developmental stages were observed. Hence, c-Maf is well-positioned to mandate liver sinusoidal identity.

### Example 5: c-Maf is a Key Determinant of Liver Sinusoidal Signature

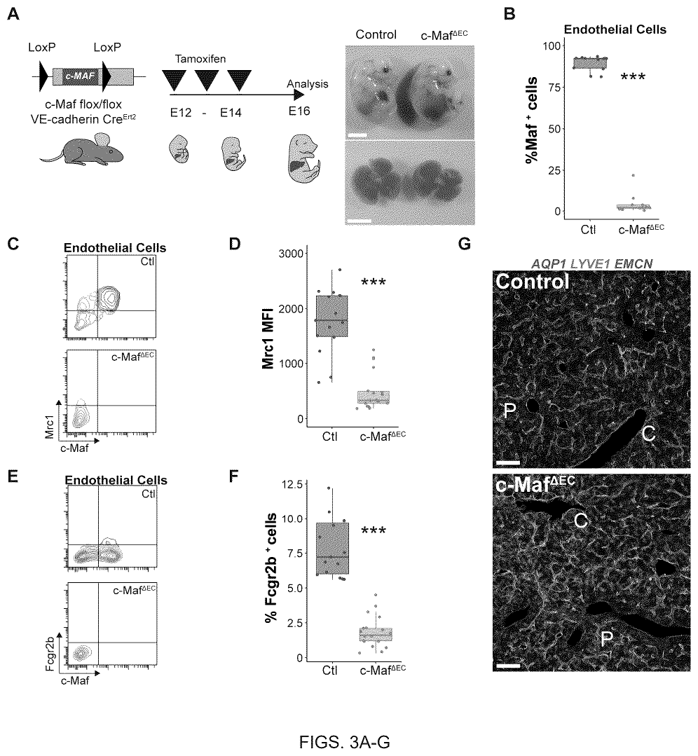

Developmental deletion of c-Maf is embryonically lethal phenotype due to a lack of erythropoiesis associated with aberrant alteration of macrophages in the blood islands of the liver, and reduced liver size (Kusakabe et al., (2011) Blood 118, 1374-1385). To circumvent this impediment and study the contribution of endothelial c-Maf in regulating sinusoidal differentiation, an inducible and tissue-specific mouse model was generated, in which upon tamoxifen treatment c-Maf was selectively and conditionally deleted in ECs (VEcadherin-Cre^{Ert2}/c-Maf^{flox/flox}) to create c-Maf^{ΔEC} mice (Wende et al., (2012) Science 335, 1373-1376). To prevent recombination in the hematopoietic lineage, c-Maf in the VEcadherin-Cre^{Ert2}/c-Maf^{flox/flox} mice was deleted by treating the pregnant mice with tamoxifen at E12 to E14 and analyzed the mice at E16 (FIG. 3A, left panel). As opposed to constitutive deletion of c-Maf, (Kusakabe et al., (2011) Blood 118, 1374-1385), embryos from c-Maf^{ΔEC} mice did not show macroscopic abnormalities, including anemia or reduced liver size (FIG. 3A, right panel), however CD45^{neg}CD31⁺ ECs (FIG. 11K) showed a reduction in c-Maf⁺ ECs (FIG. 3B), whereas CD45⁺ CD68⁺ hematopoietic myeloid cells remained unaffected (FIG. 11L).

Embryos from EC-specific c-Maf deletion showed a reduction of sinusoidal markers Mrc1 (FIG. 3C, D) and Fcgr2b (FIG. 3E, F), consistent with a critical role of c-Maf in sinusoidal identity determination. This was coupled with an increase in the venous marker Emen (FIG. 3G). Cell proliferation as measured by Ki67 did not show significant changes in the endothelial compartment (FIG. 11M). A decrease in Fractal dimension indicated that deletion of c-Maf disrupts the patterning of zonated sinusoids, (FIG. 11N). Thus, c-Maf is required during development for the phenotypic specification and formation of sinusoidal network.

### Example 6: Deletion of c-Maf Expression Prevents Sinusoidal Differentiation and Aberrantly Expands Postnatal Liver Hematopoiesis

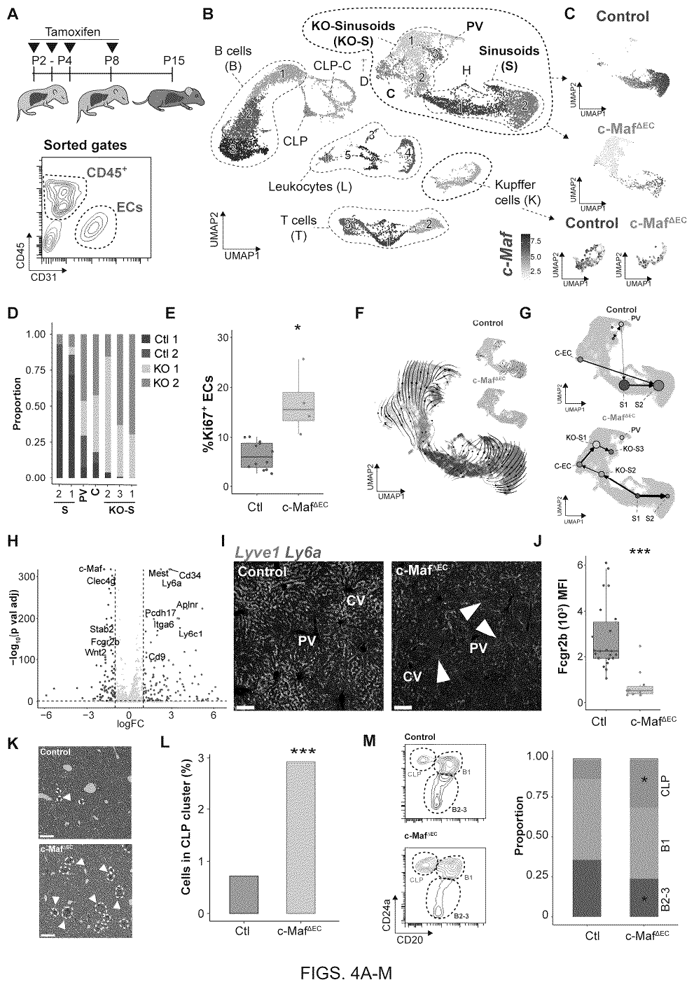

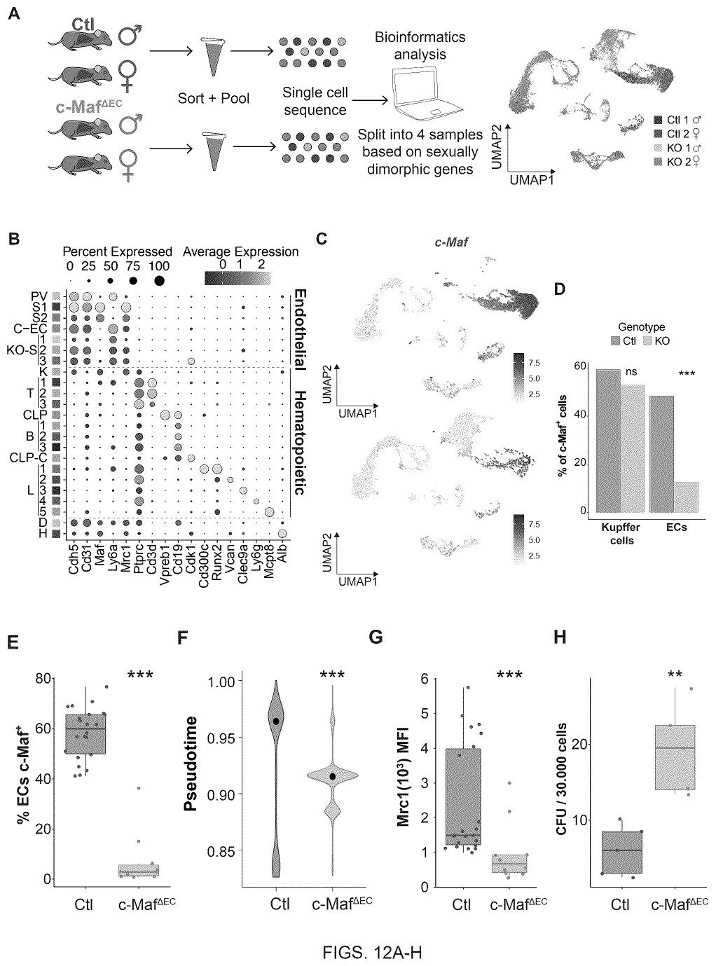

To further explore the role of c-Maf during postnatal liver development, its deletion was induced by administrating 4-hydroxytamoxifen to mouse pups and analyzing them prior to P15 (FIG. 4A, upper panel). scRNA-seq analysis of cells from the endothelial (CD31⁺ CD45^{neg}) and hematopoietic (CD3^{neg}CD45⁺) compartments were performed (FIG. 4A, bottom panel). Cells from two animals, male and female, per condition, control and c-Maf^{ΔEC}, were sorted and combined, and separated analytically based on the expression of sexual dimorphism genes (FIG. 12A). A total of 23 populations were identified, subdivided into 7 vascular endothelial sub-clusters, 14 hematopoictic sub-clusters, and two clusters of contaminant doublets and hepatocytes (FIG. 4B, FIG. 12B). Within the ECs, c-Maf expression was reduced in c-Maf^{ΔEC} cells compared to controls, but not in Kupffer cells (FIG. 4C and FIG. 12C, D). In c-Maf^{ΔEC} mice, only a small fraction of ECs (˜17.7%) retained c-Maf expression, in line with a deletion efficiency of 92.8% calculated by flow cytometry (FIG. 12E). EC clusters were observed in the c-Maf deficient mice that were absent in the WT counterparts (clusters KO-S [1-3]; FIG. 4D) suggestive of a sinusoidal differentiation defect. Notably, within the EC clusters, there was an increased proportion of proliferative Ki67⁺ ECs in the c-Maf^{ΔEC} mice (FIG. 4E).

To estimate the differentiation trajectories of ECs, RNA-velocity analysis was performed (Bergen et al., (2020) Nat. Biotechnol. 38, 1408-1414) (FIG. 4F). A distinct trajectory was observed in c-Maf^{ΔEC} ECs towards KO-S [1-3] cell clusters (FIG. 4F, inset). These differentiation trajectories were also observed when performing graph abstraction analysis to estimate transition confidence (FIG. 4G). Consistent with a differentiation defect in c-Maf^{ΔEC} liver ECs, RNA velocity-derived pseudotime analysis showed an enrichment of c-Maf^{ΔEC} cells at early pseudotime points (FIG. 12F). Analysis of the differentially expressed genes between the Control and c-Maf^{ΔEC} ECs showed decreased expression of sinusoidal genes, such as Fcgr2b, Stab2, Clec4g and Wnt2, and an increase in arterial genes, including Cd34, Ly6a, Aplnr and Cd9 (FDR<0.05, log₂FC>1; FIG. 4H). The changes in the expression of sinusoidal and arterial signatures were further confirmed by immunofluorescence analysis of Ly6a and Lyve1 staining, showing an increase in Ly6a and decrease in Lyve1 staining (FIG. 4I). Furthermore, a decrease in Fcgr2b (FIG. 4J) and Mrc1 (FIG. 12G) were shown. These results are consistent with scRNA-seq analysis of an unscheduled appearance of a unique cell cluster of ECs within the c-Maf^{ΔEC} sinusoids with higher expression of arterial genes.

The expression of an arterial phenotype is associated with emergence of hematopoietic progenitors in the fetal liver and adult bone marrow (Guo et al. (2017) J. Clin. Invest. 127, 4242-4256; Khan et al. (2016) Science 351, 176-180; Poulos et al. (2013) Cell Rep. 4, 1022-1034). Histological analysis of the liver by H&E staining showed an increase in hematopoietic cells, specifically common lymphoid progenitor (CLP) cell cluster and decrease of differentiated B2-B3 cells in c-Maf^{ΔEC} mice livers (FIG. 4K, L, M). Matrix cellulose colony-forming assays also confirmed an increase in hematopoietic progenitors as shown by a significant increase in colony forming units in the c-Maf^{ΔEC} mice (FIG. 12H). Hence, postnatal vascular c-Maf deficiency impairs the acquisition of the sinusoidal identity, promotes an arterial phenotype and pathophysiological retention of liver hematopoiesis.

### Example 7: c-Maf Orchestrates Liver Sinusoidal Program in Adult Mice

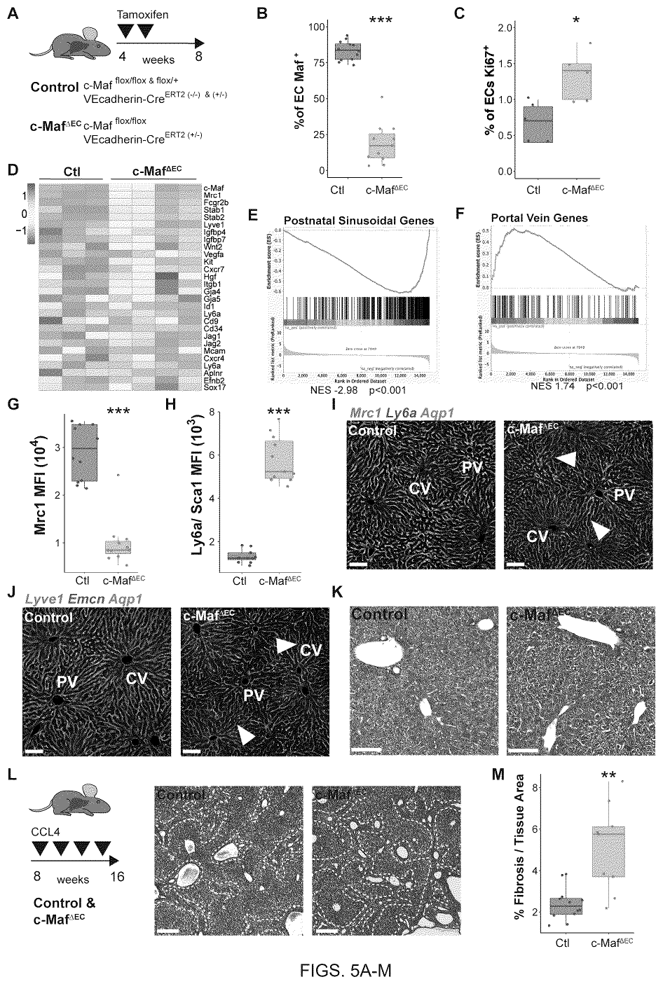

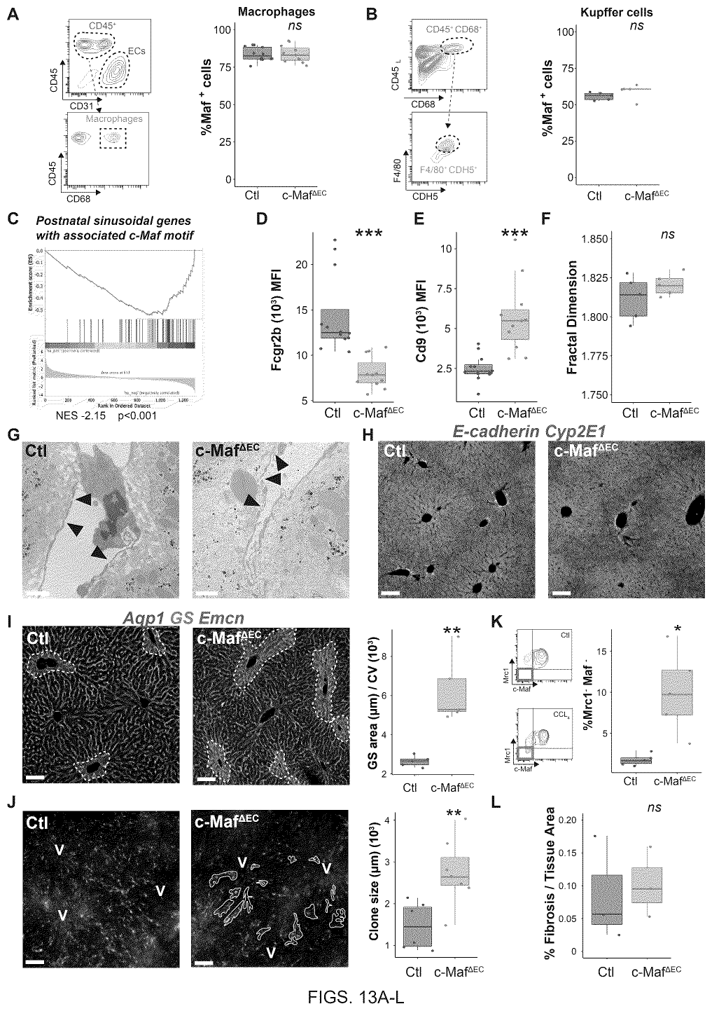

To investigate the role of c-Maf in the adult liver endothelium, c-Maf deletion was induced in ECs at postnatal week 4 and analyzed them at postnatal week 8 (FIG. 5A). c-Maf deletion was observed in 81.3% of the ECs (FIG. 5B), with no significant effect in Cd45⁺ Cd68⁺ myeloid cells (FIG. 13A), nor in Cd45⁺ Cd68⁺F4/80⁺ Cdh5⁺ Kupffer cells (FIG. 13B). Similar to what was observed at P15, there was an increase proliferative capacity of the ECs in the c-Maf^{ΔEC} mice (FIG. 5C). RNA-seq analysis showed an overall decrease in sinusoidal signature, such as Mrc1, Fcgr2b, Stab1, Stab2, and Lyve1 and angiocrine genes, including Wnt2, Hgf, Itgb1, Igfbp4, and Igfbp7 in c-Maf^{ΔEC} mice (FIG. 5D). Conversely, expression of genes involved in the Portal Vein transcriptional program was increased, including Ly6a, Cd34, Cd9, Ephb2, Gja5 and Sox17 (FIG. 5D). Employing gene set enrichment analysis (GSEA) also showed a depletion of postnatal sinusoidal genes (FIG. 5E) and an increase in Portal Vein genes (FIG. 5F).

To unravel c-Maf regulatory landscape, the position of the c-Maf DNA binding motifs were defined within open chromatin regions of liver ECs using available ATAC-seq data (Winkler et al., (2021) J. Hepatol. 74, 380-393). Next the list of genes was intersected with associated c-Maf motifs, and the sinusoidal gene list derived from the scRNA-seq data. Gene set enrichment analysis showed a decreased signature of postnatal sinusoidal genes with associated c-Maf motif in c-Maf^{ΔEC} mice (FIG. 13C). A decrease in sinusoidal Mrc1 (FIG. 5G) and Fcgr2b (FIG. 13D) was also shown and an increase in arterial Ly6a (FIG. 5H) and Cd9 (FIG. 13E). Upon c-Maf deletion, immunofluorescence analysis indicated the decrease of Mrc1 and concomitant increase of the arterial marker Ly6a in liver sinusoids (FIG. 5I). Moreover, expression of other adult sinusoidal markers, including Lyve1 were disrupted, along with increased expression of the venous marker Emen in the vicinity of the Central Vein (FIG. 5J). However, deletion of c-Maf in adults with already established vascular networks did not alter fractal dimension patterning (FIG. 13F).

Analysis of the microstructure of the sinusoids by electron microscopy did not show absence of fenestrations after c-Maf deletion, suggesting that other pathways might control this unique structural remodeling (FIG. 13G). Notably, analysis of the zonation markers E-cadherin and Cyp2E1 also did not reveal changes in the portal to centro-lobular zonation gradient (FIG. 13H). However, an expansion of glutamine synthetase-expressing hepatocytes within the c-Maf^{ΔEC} mice was observed, with an increase staining of the venous marker Emcn (FIG. 13I), suggestive of mild disruption of liver co-zonation. Loss of sinusoidal identity can potentially arise from two distinct scenarios: i) expansion of the Portal Vein or ii) activation of arterial signatures within the sinusoids. Previous results in postnatal development suggested the latter scenario, together with increased in the proliferative phenotype. To test this hypothesis, confetti c-Maf^{ΔEC} mice was generated to examine clonal expansions in the liver. An increased size of sinusoidal clones was observed, supporting the previous observations (FIG. 13J). Thus, c-Maf deletion in adult liver sinusoids results in the loss of sinusoidal identity with acquisition of Portal Vein markers and a mild disruption of liver co-zonation.

Loss of both liver co-zonation and sinusoidal identity triggers liver fibrosis (Desroches-Castan et al., (2019a) Hepatology 70, 1392-1408). Hence, it was studied whether induction of fibrosis resulted in loss of c-Maf positive cells. Liver fibrosis was induced using biweekly doses of 25% carbon tetrachloride (CCl₄ upon c-Maf deletion) for 1 month. In line with c-Maf capable of determining sinusoidal identity, induction of fibrosis resulted in an increase of Mrc1^{neg}/c-Maf^{neg} ECs (FIG. 13K). Although no fibrosis was observed in basal conditions in the c-Maf^{ΔEC} mice (FIG. 5K, and FIG. 13L), CCl₄ treatment increased fibrotic area compared to controls (FIG. 5L, M). Therefore, loss of sinusoidal identity postnatally is not sufficient for induction of liver fibrosis in adult mice but increases predisposition towards healing by fibrosis upon chemical insult. Hence, c-Maf is required for the maintenance of liver sinusoidal identity and for restoring fibrosis-free homeostasis upon chemical insult in adult mice.

### Example 8: scRNA-Seq of Human Liver ECs Informs of Signature-Based Isolation Strategy

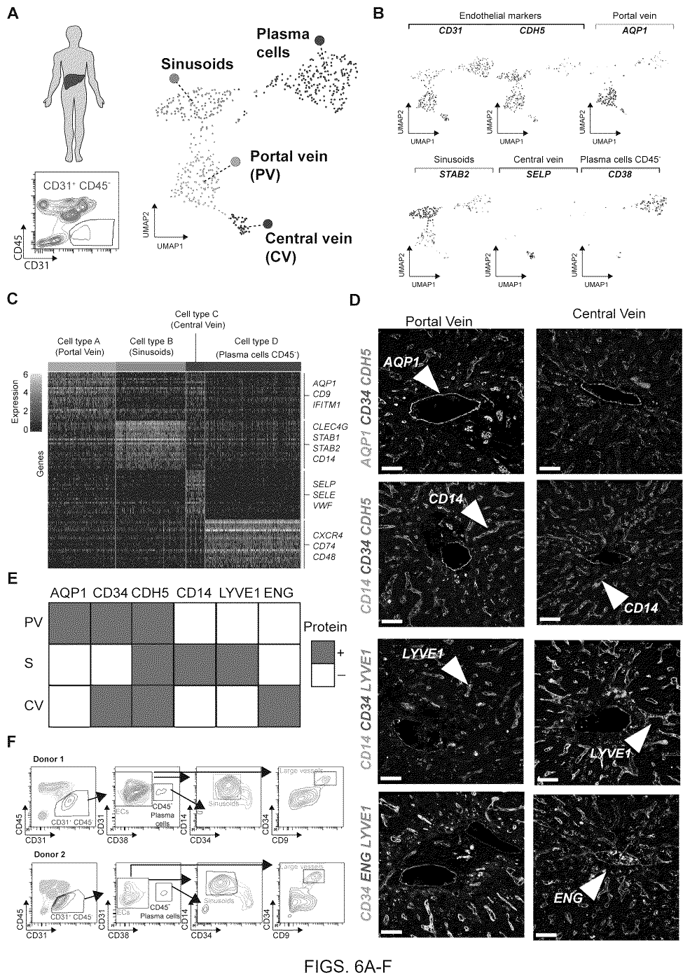

scRNA-seq of liver sinusoids demonstrate co-zonation with hepatocytes (Halpern et al., (2018) Nat. Biotechnol. 36, 962-970.; MacParland et al., (2018) Nat. Commun. 9, 4383). Here, scRNA-seq analysis of the CD45^{neg}CD31⁺ population in human adult liver was performed to resolve cell heterogeneity. Four distinct cell clusters were observed, of which the clusters representing Sinusoids, Portal Vein and Central Vein were assigned to the EC compartment based on the expression of CD31, CDH5, AQP1, STAB2 and SELP (FIG. 6A, B). Notably, plasma cells were identified as CD45^{neg}CD31⁺ and CD38 (MacParland et al., (2018) Nat. Commun. 9, 4383) (FIG. 6A, B). The Sinusoid and Portal Vein clusters expressed well-established marker genes (FIG. 6C) that enabled their classification based on prior studies (Halpern et al., (2018) Nat. Biotechnol. 36, 962-970.; MacParland et al., (2018) Nat. Commun. 9, 4383). To resolve the identity of the Central vein cluster, differential expression between Central and Portal Vein clusters were performed (FIG. 14A), which warranted its classification based on ENG and RSPO3 angiocrine expression. Furthermore, while large vessels are CD34⁺, the Portal Vein is the only AQP1⁺ human vascular population (FIG. 6D, E). Notably, the expression of CD14 and LYVE1 was associated with sinusoidal ECs, while endoglin (ENG) was observed exclusively in Central Vein (FIG. 6D, E). Accordingly, a flow cytometry strategy was developed that allowed to identify human liver ECs as CD45^{neg}CD31⁺ CD38^{neg} cells, of which the sinusoids were phenotypically defined as CD45^{neg}CD31⁺ CD38^{neg}CD14⁺ CD34^{low}, and large vessels (Portal and Central Veins) as CD45^{neg}CD31⁺ CD38^{neg}CD14⁺ CD34^{high}CD9⁺ ECs (FIG. 6F). These results expanded the previous description of the molecular features of the human liver endothelium and could potentially serve as a reference dataset for future analysis.

Importantly, analysis of a human fetal liver at 17 weeks of development showed a different pattern from the adult. Similar to mouse development, the Portal Vein was phenotypically differentiated early in development, with the absence of the sinusoidal marker LYVE1, while the Central Vein remained LYVE1⁺ (FIG. 14B). The sinusoids were defined as CD14⁺ in the human fetal liver at 17 weeks, recapitulating previous observations at week 20 (Couvelard et al., (1996) Blood 87, 4568-4580) (FIG. 14C). Unlike in adult liver CD31/CD45 gating strategy, no plasma cells marked by CD45^{neg}CD31⁺ CD38⁺, were observed during fetal development (FIG. 14C). Therefore, scRNA-seq analysis of sorted CD31⁺ CD45^{neg} revealed specific markers for precise gating of adult liver ECs. The proposed strategy prevents inclusion of plasma cells into the EC gate, improving the purity of the EC population for downstream analysis.

### Example 9: Enhanced Expression of c-Maf Induces the Human Sinusoidal Transcriptional Program In Vitro

Next the human scRNA-seq data was leveraged to explore the role of c-Maf in human ECs enforced specification into sinusoidal signature. Notably, human liver sinusoids showed higher expression levels of c-Maf compared to the Portal or Central Veins (FIG. 7A). To test whether c-Maf has the capacity to induce a sinusoidal program in human ECs, a doxycycline inducible lentiviral system was used to overexpress c-Maf in human Umbilical Vein ECs (HUVECs) that represent generic vessels (FIG. 7B).

Human liver sinusoids are known to co-express microvascular markers, including CD36, CD26 (Fukui et al., (1990) Cell Struct. Funct. 15, 117-125; Strauss et al., (2017) Sci. Rep. 7, 44356), and CD14 (Couvelard et al., (1996) Blood 87, 4568-4580); MacParland et al., (2018) Nat. Commun. 9, 4383; Strauss et al., (2017) Sci. Rep. 7, 44356). Enhanced expression of c-Maf in HUVECs led to increase expression of CD36 by immunofluorescence and morphological changes (FIG. 7C and FIG. 14D, E), and emergence of double positive CD36⁺/CD26⁺ population comprising 54.76%±9.36 (mean±standard deviation) of EC population (FIG. 7D). The CD36⁺ CD26⁺ population was termed as Induced Liver Sinusoidal ECs (ILSECs). RNA-seq analysis of the iLSECs showed an enhanced expression of multiple liver sinusoidal markers (FDR<0.05) compared to control cells (FIG. 7E), which was further supported by gene set enrichment analysis (FIG. 7F), although not all sinusoidal genes, such as Fegr2b, were induced. Remarkably, human liver sinusoidal markers CD14 and MRC1 (FIG. 14F, G) were also induced in iLSECs overexpressing c-Maf (FIG. 14H). Thus, enforced c-Maf expression is sufficient to induce activation of a pro-sinusoidal transcriptional program and sinusoid phenotype in human ECs in vitro.

To determine if iLSECs support hepatocyte function, a co-culture system of iLSECs was designed with human adult primary hepatocyte aggregates (Song et al., (2015) Sci. Rep. 5, 16884) (FIG. 7G). Electron microscopy showed ECs from control and iLSECs interacting with the hepatocyte aggregates at day 7, displaying cytoplasmic fenestration gaps in the iLSECs, but not in the controls (FIG. 14I). In order to assess hepatocyte function, the expression of CYP1A2, a key enzyme in liver metabolism, and the secretion of albumin during 28 days of co-culture were quantified. Contrary to mock-transduced control ECs, ILSECs sustained long-term hepatocyte function, as demonstrated by CYP1A2 expression (FIG. 7H) and maintenance of albumin production (FIG. 7I). Thus, enhanced expression of c-Maf is sufficient for induction of liver sinusoidal identity and enables long-term sustainability of hepatocyte function. Therefore, c-Maf plays a key role in specifying sinusoidal identity and function.

What is claimed is:

1. An isolated population of induced liver sinusoidal endothelial cells (ILSECs), wherein isolated population comprises iLSECs that express a c-Maf protein from an exogenous nucleic acid and express at the cell surface at least two markers selected from the group consisting of CD26, CD36, and CD14.

2. The isolated population of iLSECs of claim 1, wherein the iLSECs express CD26, CD36, and CD14.

3. The isolated population of iLSECs of claim 1 or 2, wherein the iLSECs additional express MRC1 at the cell surface.

4. The isolated population of iLSECs of any one of claims 1-3, wherein the exogenous nucleic acid comprises a viral vector such as a lentiviral vector or AAV vector.

5. The isolated population of iLSECs of any one of claims 1-3, wherein the exogenous nucleic acid comprises a modified RNA (modRNA).

6. The isolated population of iLSECs of any one of claims 1-5, prepared by introducing the exogenous nucleic acid into a population of endothelial cells and expressing the c-Maf protein in the population of endothelial cells in culture.

7. The isolated population of iLSECs of claim 6, wherein the population of endothelial cells is a population of endothelial cells without a liver signature selected from the group consisting of human umbilical vein endothelial cells (HUVECs), adipose-derived endothelial cells, organ-specific endothelial cells, endothelial stem cells, and endothelial progenitor cells, and wherein the endothelial cells are autologous or allogeneic.

8. The isolated population of iLSECs of claim 7, wherein the organ-specific endothelial cells are selected from the group consisting of heart-specific endothelial cells, muscle-specific endothelial cells, kidney-specific endothelial cells, testis-specific endothelial cells, ovary-specific endothelial cells, lymphoid-specific endothelial cells, pancreas-specific endothelial cells, brain-specific endothelial cells, lung-specific endothelial cells, bone marrow-specific endothelial cells, spleen-specific endothelial cells, large intestine-specific endothelial cells, small intestine-specific endothelial cells, and ovary or testicular endothelial cells.

9. The isolated population of iLSECs of any one of claims 1-8, wherein the c-Maf protein is human c-Maf or mouse c-Maf.

10. The isolated population of iLSECs of any one of claims 1-9, wherein the isolated population of iLSECs supports a co-culture with hepatocytes for at least 20 to 30 days.

11. A method of producing an isolated population of induced liver sinusoidal endothelial cells (ILSECs), comprising
providing a population of endothelial cells,
expressing a c-Maf protein from an exogenous nucleic acid in the population of the endothelial cells in culture,
selecting a population of cells that express at the cell surface at least two markers selected from the group consisting of CD26, CD36, and CD14, thereby obtaining said isolated population of iLSECs.

12. The method of claim 11, wherein the cells selected express at the cell surface all three markers of CD26, CD36, and CD14.

13. The method of claim 11 or 12, wherein the cells selected also express MRC1 at the cell surface.

14. The method of any one of claims 11-13, wherein the exogenous nucleic acid comprises a viral vector such as a lentiviral vector or AAV vector.

15. The method of any one of claims 11-13, wherein the exogenous nucleic acid comprises a modified RNA (modRNA).

16. The method of any one of claims 11-15, wherein the population of endothelial cells is a population of endothelial cells without a liver signature selected from the group consisting of human umbilical vein endothelial cells (HUVECs), adipose-derived endothelial cells, organ-specific endothelial cells, endothelial stem cells, and endothelial progenitor cells, and wherein the endothelial cells are autologous or allogeneic.

17. The method of claim 16, wherein the organ-specific endothelial cells are selected from the group consisting of heart-specific endothelial cells, muscle-specific endothelial cells, kidney-specific endothelial cells, testis-specific endothelial cells, ovary-specific endothelial cells, lymphoid-specific endothelial cells, pancreas-specific endothelial cells, brain-specific endothelial cells, lung-specific endothelial cells, bone marrow-specific endothelial cells, spleen-specific endothelial cells, large intestine-specific endothelial cells, small intestine-specific endothelial cells, and ovary or testicular endothelial cells.

18. The method of any one of claims 11-17, wherein the c-Maf protein is human c-Maf or mouse c-Maf.

19. The method of any one of claims 11-18, wherein the cells expressing c-Maf are cultured for 6-8 days before selecting for cell surface markers.

20. A method of maintaining hepatocytes comprising:
culturing hepatocytes in the presence of an isolated population of iLSECs according to any one of claims 1-10.

21. The method of claim 20, wherein the culturing is done for at least 20 days to at least 30 days, e.g., at least 28 days.

22. The method according to any one of claims 19-21, wherein the culturing is carried out in a bioreactor, a tissue culture plate or a microfluidic device.

23. A method of treating a damaged liver or a liver disease in a subject comprising administering to the subject an isolated population of iLSECs according to any one of claims 1-10.

24. The method of claim 23, further comprising administering to the subject hepatocytes together with the isolated population of iLSECs.

25. The method of claim 23 or 24, wherein the administering is achieved by a method selected from the group consisting of surgical or catheter implantation, subcutaneous injection, and infusion through an intravascular route.

26. The method of any one of claims 23-25, wherein the isolated population of iLSECs is administered to a site in the omentum or in the liver under the liver capsule.

27. The method of any one of claims 23-26, wherein the liver disease is selected from the group consisting of acetaminophen toxicity, acute liver failure, alcoholic liver disease, liver cancer, cirrhosis, liver cyst, non-alcoholic fatty liver disease (NAFLD), and liver fibrosis.

28. A method of treating a subject suffering from a liver disease comprising:
administering to the subject hepatocytes which have been co-cultured with an isolated population of iLSECs according to any one of claims 1-10.

29. The method of claim 28, wherein the administering is achieved by a method selected from the group consisting of surgical implantation, catheter implantation, subcutaneous injection, and infusion through an intravascular route.

30. The method of claim 28 or 29, wherein the hepatocytes are administered to a site in the omentum or in the liver under the liver capsule.

31. The method of any one of claims 28-30, wherein the liver disease is selected from the group consisting of acetaminophen toxicity, acute liver failure, alcoholic liver disease, liver cancer, cirrhosis, liver cyst, non-alcoholic fatty liver disease (NAFLD), and liver fibrosis.

32. The method of any one of claims 28-31, wherein prior to the administering, the hepatocytes have been co-cultured with the isolated population of iLSECs for at least 3-4 weeks.

33. The method of any one of claims 28-32, wherein the hepatocytes have been co-cultured with the isolated population of iLSECs in a bioreactor, a tissue culture plate or a microfluidic device.

34. The method of any one of claims 28-33, wherein the hepatocytes have been co-cultured with the isolated population of iLSECs in a decellularized matrix, 3D scaffold, or bioengineer matrix for the transplantation.

35. The method of any one of claims 28-34, wherein the iLSECs from the co-culture are administered with the hepatocytes.
